(19) United States
(12) Patent Application Publication
(10) Pub. No.: US 2023/0245763 A1
(43) Pub. Date: Aug. 3, 2023

(54) SYSTEM FOR FINDING AND ENROLLING INDIVIDUALS IN PROGRAMS REQUIRING DETERMINATION, CERTIFICATION AND REDETERMINATION CHRONICALLY

(71) Applicant: SVENSK HEALTH SYSTEMS LLC., Chicago, IL (US)

(72) Inventors: Jacob A. Miller, Chicago, IL (US); Andrew Madigan, Chicago, IL (US)

(21) Appl. No.: 18/153,798

(22) Filed: Jan. 12, 2023

Related U.S. Application Data
(60) Provisional application No. 63/298,680, filed on Jan. 12, 2022.

(51) Int. Cl. G16H 40/20; G16H 10/60; G16H 80/00

(52) U.S. Cl. CPC G16H 40/20; G16H 10/60; G16H 80/00

(57) ABSTRACT

A method is provided for contacting a member of a group who must participate in an annual recertification of a process for securing benefits. The method includes the storage of contact information for reaching the member and, should contact be thwarted, means for scouring databases, public and private, to find better contact data, until the member can be reached or otherwise dismissed from the program. Upon contact, the member is invited to participate in an interview, either telephonically or via computer or computerized mobile device, to provide information that will be used to automatically fill in appropriate forms, the appropriateness determined through the interview, to thereby enroll the member in particular programs, such as Medicaid. The method allows the member to utilize any means of communication to complete the process or change the means of communication, including telephonic and computer or mobile means, at any time, without losing the member's place in the process or jeopardizing disclosure of secure data.

### FIELD OF THE INVENTION

The present invention concerns a self-pacing automation of the process of determination, redetermining (generally “determination”) or certification or recertifying entry into any type of regulated program; along with a system of forcing the determination process where recalcitrance or reticence is detected. More particularly the present invention concerns a system of finding, informing, recruiting and securing an individual's participation in programs such as Medicaid to the benefit of the individual.

### BACKGROUND OF THE INVENTION

There is a real and existing critical challenge regarding the manner in which healthcare, and particularly medical benefits, patient wellness and access to crucial local, state and federal coverage are enrolled, administered, maintained and kept current. Particularly, healthcare has always been behind the curve from a technology standpoint, but this is even more acute in the Medicaid space, specifically in the determination, redetermination/recertification process involved with Medicaid benefits and identification and enrollment for Supplemental Security Income (SSI) and Social Security Disability (SSDI) benefits for Medicaid beneficiaries. The purpose behind the present invention is to utilize a system or method that employs technology as a component to improve and transform the determination process especially but not limited to the methodology within the Medicaid space. This invention is also aimed at facilitating Managed Care Organizations (MCO's) ability to locate, engage with, recruit and simplify the determination process for individuals.

What's the difference between Medicare and Medicaid? According to the US Department of Health and Human Services (HHS), Medicare is federal health insurance for anyone age 65 and older, and some people under 65 with certain disabilities or conditions. Medicaid is a joint federal and state program that gives health coverage to some people with limited income and resources.

One goal of the present invention is to increase the number of people that will go through and complete the redetermination process. This is accomplished by automating the overall data collection process and in the manner in which prospects are found, location and personal communication is updated in a database and updated in the forms needed to redetermine. Further means to accomplish the goal is to continually encourage applicants to complete the redetermination process, including, making it easier for applicants or members to use one of a number of data input means and then also to be able to seamlessly move from one input means to another while also facilitating the data collection by providing redetermination forms in a simple, more condensed format and by pre-populating the forms with the individual's currently known information for easy confirmation or updating.

Medicaid renewal, also called Medicaid redetermination or recertification, is a necessary part of continuing to be a Medicaid beneficiary (also referred to herein and in the industry as “members”), regardless of whether one receive benefits through the regular state plan, or long-term home and community-based services (HCBS) via a Medicaid waiver, or are on nursing home Medicaid. The Medicaid redetermination process ensures one is still eligible to receive Medicaid benefits, and in the case of many seniors and disabled persons, continue to receive Medicaid-funded long-term services and supports. Medicaid has income and asset (resource) limits for Medicaid beneficiaries and the Medicaid agency wants to ensure that the individual continues to fall under those financial limits.

Medicaid beneficiaries must, continually, report any change in income or assets, even if it is not time for renewal of Medicaid benefits. This is because circumstances, such as an increase in income or an inheritance, can result in one being found ineligible to continue to receive Medicaid benefits. Most states allow between 10 and 30 days to report any such changes. Failure to do so can result in the loss of all Medicaid benefits and require the repayment of services and/or benefits which Medicaid has paid; failure to make such reports can result in fines and imprisonment as well.

In Illinois for example, there are several different Medicaid programs for which residents may participate in and be eligible. Bach of these programs has slightly different eligibility requirements and benefits, which must be read and understood prior to enrollment therein. Additionally, there are complications regarding a participant's eligibility for such programs, including (but not limited to) marital status and age and it is often found that the state offers different channels towards eligibility depending on eligibility; choosing the proper channel being problematic. Regardless of the program, Medicaid determination and/or recertification, is a necessary part of being a Medicaid beneficiary.

Furthermore, Medicaid enrollment and renewal processes are not consistent across states and Medicaid groups. All of this is a recipe for complications and losses for any individual who does not recognize the programs for which it is eligible and to which it can be entitled; loss of which can result in missing benefits and having financial and health related consequences.

The determination and redetermination processes are currently conducted in one of three ways: 1) by telephone interview, 2) by mail and 3) by in-person interview. Often members and prospective members are notified by mail regarding the need of and requirements for enrollment or redetermination; which, as can be imagined can be rife with problems, such as lost mail, undelivered mail, stolen mail, language issues and cognitive issues all related to the receipt of the Medicaid mailing. These issues are exasperated when the member or prospective member is elderly or mentally, physically or medically disabled or challenged. Often such eligibility and benefit redeterminations occur once annually. The specifics of the process vary state by state and even by provider/administrator. It has been found that a common weakness in the process is that members or prospective members cannot be located, and/or forget to follow through with the redetermination process and/or simply do not care enough to make the effort to proceed with the process and/or do not have the wherewithal or assistance to understand the process and thus fall out of the determination or fail to determine or redetermine.

In the case of telephonic and in-person interviews, participants are typically sent a letter, in the mail, telling them to expect a call at a certain date and time. Many, who need assistance with the process, are often at a severe cognitive disadvantage and can and do miss appointments, with the consequence of loss of benefits. Typically, the Managed Care Organization (MCO) is aware of the process at least 60 to 90 days prior to the annual deadline; but there may be circumstances wherein participants can wind up having as few as 30 days to respond to an appointment letter, complete and return the form, or advise why they either cannot keep the appointment or are having trouble filling out the form. If they do not satisfy the process requirements, then they could lose Medicaid eligibility at that time and would need to reapply to determine eligibility, which is time-consuming and tedious and laden with hurdles for many possible participants.

Oftentimes, a Medicaid recipient may be in tolerably good health, at the time of the process, and may ignore the need to determiner or redetermine, or the determinant feels that it does do not need any medical or healthcare coverage and therefore falls out of the program. This is often an error and can cause delays in coverage when a need does suddenly occur.

Some purposes behind the present invention then, and as described in the preferred embodiment, is to serve the public medical insurance space and related commercial functioning behind this space in a convenience, efficient, secure, and optimal manner so Medicaid benefits are secured and/or continue from certification period to certification period. The invention is also applicable to other areas where chronic enrollment or re-enrollment is required.

Another critical characteristic of the present invention is found in its ability to simplify and greatly facilitate the ability of members to engage in the redetermination process and easily enter, update and transmit their required information and data by being able to go from using a virtual agent or voice chat to an online web form with a virtual text chat feature and even back to a human assistant seamlessly and without losing any data input. A particular unique feature of this invention is that it allows the member to start the process through one of the means available, stop at some point and then reconvene using the same means or transferring the process to another means available through the system without missing a beat. Unique to this system is that the member can switch between a virtual agent (using artificial intelligence) on the telephone, to an online web form with a virtual text chat assistant to an old-school human assistant. Another useful feature of this inventive system is that in many cases the redetermination form is pre-populated with the member's then known information; which can be uploaded during the redetermination process as the member is walked through the data fields so as to confirm or update the member's information.

Another aspect of the present invention's redetermination processing is that the various data collection means are designed to be compliant with the Americans with Disabilities Act (ADA). ADA compliance, by aspects of the present invention, is found regardless of which of the different means to enter data provided by the invention are being used by the member, and then, any of the other means to which the member can switch, continues to be ADA compliant regardless of the stage of the redetermination process that user is in and regardless of the number of transitions from one data entry means to another.

In another aspect of the invention, the scouring systems will be utilized to review any changes to the Medicaid program and institute those changes in the questionnaires and forms utilized to assess the candidate and enroll the applicant.

In another aspect of the invention, the contact with a member is automated, taking last know contact information to reach out to the member, and if the process is advised that contact was not made, the process uses public and private databases to find and test newly found contact information until the member is reached and redetermination is started, or the member opts out of the process. The system is designed to continue until contact is made and confirmed and contact information is updated to facilitate the following years redetermination contact process. A relentless search to find members and complete the redetermination process is made hereby, such that no eligible member or prospect is left behind. Additionally, in some embodiments, should it be discovered that a member has moved to a different state, and therefore a different jurisdiction, the present invention can notify an MCO in that jurisdiction and provide the latest contact information for the member, such that the member can be enrolled in its new jurisdiction.

In yet another aspect of this invention, there is an ability to reward members who successfully completes the redetermination process. In one means of reward, a gift card or the like can be provided to the member when the process is completed. The amount of the gift card can be conditioned on the responsiveness of the member or dependent on other factors such as age, living conditions, abode as well as other factors or criteria. In some embodiments of the present invention, a Heath Risk Screening (HRS) can also be conducted during the initial interview process, including a questionnaire about the member's health history, including family history and to determine any existing healthcare conditions, so as to not only enroll or conduct the redetermination, but also to begin immediately the task of getting the member the proper health or well care needed. It will be seen that when family members are part of the process, the health history of one family member can be applied to the entire family to better anticipate the health needs of any one of the related members.

Therefore, it will be seen that there is a real, current and critical need for a method that can be employed, administered and utilized easily and electronically, such as over the Internet and on computer systems such as desk top or laptop computers or preferably through a mobile application over a mobile device, that better ensures participation by all eligible persons in a program for their benefit. The invention provides a structured means to find and notify participants and potential participants and then guides those persons through the process to secure such participants benefits to which they are eligible and of which they are in need; update databases to make note of the participant, their preferred method of communication and enrollment in the processes and their eligibility for their current benefits and by reaching certain standards (such as age, marital status, and location) for benefits that become available in the next and future enrollment periods. The invention thus is not the mechanized use of present processes to fill out forms, but is instead an improvement on systems, including computerized systems, to find, contact, advise, enroll, store data, consider benefits, to advise of changes in the future and continually update itself to more effortlessly repeat these processes chronically as data is acquired and manipulated by the system for these purposes.

Although the disclosure herein, and the embodiments set forth, describe a particular application in the Medicaid arena, the present invention may be altered or configured for adaptation to other uses in the medical insurance industry or even other areas or types of insurance, government programs (such as licensure and relicensure) or commercial areas. Therefore, although the current invention is described herein particularly in relation to Medicaid, this invention may be adapted for other applications that requires redetermination as well as situations where customers, members, subscribers, applicants and the like are required to renew or resubmit information or documentation on a periodic basis so as to maintain their status, retain benefits or rights or otherwise continue being a customer, participant, member, subscriber, applicant and the like of an association, organization, club, group, program or entity. In fact, the current invention can be adopted to be used for anything that requires a renewal, resubscription or some sort of reporting to maintain a status, right or license. One other governmental program that is akin to Medicaid and related thereto is SSI benefits which also requires redetermination in order to maintain these benefits. For completeness, it will be understood that Medicare programs also require participants to fulfill redetermination in order to maintain benefits.

Objects and advantages of the present invention will become apparent as the description proceeds.

### SUMMARY OF THE INVENTION

In accordance with the present invention, a method of finding and enrolling or re-enrolling a person in a specific desirable program, using telephonic, computer and electronic communications means, is provided. The method comprises the steps of providing means for communication with a candidate or person assumed to meet requirements for admission to the desirable program and if communication is not made, automatically using a search program and computer to find personal contact information of the candidate to then reattempt communication therewith. Storing the personal contact information of the candidate in a secured rewritable and searchable computer database having a calendar for use now and for finding the candidate again after a preset time for the next timely period and then utilizing the personal contact information to communicate with the candidate. Determining, through the interrogation of the candidate, if the candidate is eligible for the desirable program, and if so, renaming the candidate an applicant. Directing the applicant to an appropriate enrollment means for providing information for enrollment in the desirable program and securing enrollment data, through the enrollment means, from the applicant. Then storing the enrollment data in the secured rewritable and searchable database, and associating the data with the personal contact information for the applicant as applicant data for use in enrollment and for later contact. Automatically, by allowing the computer to use the applicant data, merging the applicant data to a precise computer chosen application form to create an application for enrollment in the specific desirable program and submitting the application form for enrollment. Then updating the applicant data within the data storage device or a cloud storage service, and associating the data with the calendar to automatically contact the applicant, using the updated applicant data, within a specified time period for re-enrollment.

It will be understood, by persons having ordinary skill in the art, that enrollment data can be secured via a telephone conversation, a questionnaire on a mobile device or a questionnaire on a personal computer. Further, securing enrollment data can be started on any one of a telephone conversation, a questionnaire on a mobile device or a questionnaire on a personal computer and completed, later, on any one or more of a telephone conversation, a questionnaire on a mobile device or a questionnaire on a personal computer.

The primary focus of the method is enrollment into Medicaid; however, it will be understood that the method is applicable to other programs necessitating that a person re-enroll or apply on a chronological basis. Such programs as general medical insurance, licensure recertification (including, but not limited to professional licenses, driver's licenses, firearm licenses), memberships, annual donations and tax reporting, can be facilitated using the system of the present invention.

Use of the method can proceed, where the computer noted can include any electronic data entry and manipulation devices including one or more of a personal mobile device, a mobile telephone, an electronic pad, an electronic tablet, a personal computer, a laptop computer or any other data entry device and components thereof, including software and peripherals.

Enrollment data, for eligibility, is created by the applicant answering a series of questions, the answers being automatically recorded in the secure database.

The method includes means wherein when an attempt to communicate with a candidate is unsuccessful, the process automatically uses means to search public and private databases to find updated contact information and then uses the updated contact information to again attempt to communicate with the candidate; this process continuing until communication is made.

As some programs are limited to certain jurisdictional bodies, such as individual states, in some embodiment the process includes means to notify a provider in a second jurisdiction of a candidate who has moved to the second jurisdiction from the particular jurisdiction for enrollment of the member therein.

The questionnaire used to make assessments can, in preferred embodiments, include a Health Risk Screening to help assess the candidate's present health and needs during enrollment.

The method provided includes the step of utilizing the means for communication and storage to periodically scour the regulations of the specific desirable program to allow for automated changes in one or more of the questionnaire, forms and enrollment data as required for enrollment relative to any changes in laws or regulations.

In a preferred embodiment, then, the present invention, is a method of finding and enrolling or re-enrolling persons into Medicaid. The method uses telephonic, computer and electronic communications means, and comprises the steps of communicating with a candidate or person assumed to meet requirements for admission to Medicaid via a telephone conversation, a questionnaire on a mobile device or a questionnaire on a personal computer and if communication is not made, automatically using a search program and computer to find personal contact information of the candidate to then reattempt communication therewith. Storing the personal contact information of the candidate in a secured rewritable and searchable computer database having a calendar. Utilizing the personal contact information to communicate with the candidate and determining if the candidate is eligible for Medicaid, and if so, renaming the candidate an applicant.

The method then directs the applicant to an appropriate enrollment means for providing information for enrollment in Medicaid and securing enrollment data, wherein securing enrollment data can be started on any one of a telephone conversation, a questionnaire on a mobile device or a questionnaire on a personal computer and completed, later, on any one or more of a telephone conversation, a questionnaire on a mobile device or a questionnaire on a personal computer, from the applicant, and storing the enrollment data in the secured rewritable and searchable database, and associating the data with the personal contact information for the applicant as applicant data. Automatically, by allowing the computer to use the applicant data, merging the applicant data to a precise computer chosen application form to create an application for enrollment in the specific desirable program and submitting the application form for enrollment. Then updating the applicant data and associating the data with the calendar to automatically contact the applicant, using the updated applicant data, within a specified time period for re-enrollment.

The method includes using any electronic data entry and manipulation devices including one or more of a personal mobile device, a mobile telephone, an electronic pad, an electronic tablet, a personal computer, a laptop computer or any other data entry device and components thereof, including software and peripherals. Enrollment data is created by the applicant answering a series of questions, the answers being automatically recorded in the secure database.

In a preferred embodiment, when an attempt to communicate with a candidate is unsuccessful, the process automatically uses means to search public and private databases to find updated contact information and then uses the updated contact information to again attempt to communicate with the candidate; this process continuing until communication is made.

### ADDITIONAL DESCRIPTION OF THE PREFERRED EMBODIMENT OF THE PRESENT INVENTION

Determination of access to Medicaid is age, income and location dependent. Redeterminations, that is renewal of the programs previously enrolled in, are required to be conducted every 12 months under the law, as specially addressed in the Code of Federal Regulations (CFR), at 42 CFR § 435.916. Without fulfilling the redetermination step, the recipient (member) will lose Medicaid coverage and will not have the additional health care coverage provided by Medicaid. It has been found that MCO's have a redetermination success rate of less than 60% because, among other reasons, they utilize antiquated methods, such as paper mail and telephone calls, to try and secure redetermination of their clients. The present invention utilizes means and methods whereby a third party will be contracted by the MCO to provide the best-in-class technology to determine a member's contact information and then to update that member's information, as well as to provide support to the member in the redetermination process on behalf of the MCO's and thereby secure the redetermination for the member in their program.

The present invention utilizes means and methods, such as through the implementation of software to identify and then perform computer online searches for information necessary to contact the current Medicaid members. The method utilizes various currently available public information resources upon which to perform the searches on individuals, in order to obtain current information such as: telephone number(s), physical address and email addresses; also known as personal contact information. The invention uses such resources as Lexis/Nexus® and SmartDial® as well as other Internet resources and public records; this listing not being meant to be comprehensive. The specific entity or technology used to perform this investigative step, or search function, is not critical or meant to be limiting to the present invention—it is merely the means for performing this step; the manner of completing this step is not limited.

It will be understood that the method of the current invention will use proprietary technology, in the form of software, to automatically find and contact the member. In one preferred embodiment, the mobile telephone number of the member will be found and the member will be contacted, preferably through the delivery of a text message, via the Medicaid member's mobile telephone number. The text message will include information for the member but most importantly will provide the member with a link so that the member can access a virtual agent who can then aid the member through the redetermination process, step by step. The end result will be that the virtual agent will gather the appropriate redetermination information and aspects of the present invention will then populate the appropriate documents needed for determination and redetermination. In some states, the appropriate document is the Application for Benefit Eligibility form (ABE), but persons having ordinary skill in the art will understand that a database, associated with the present invention, can be searched to determine the appropriate application or other form necessary for the particular applicant, based on such things as name, age, location and income, among others. The invention, upon filling in the application papers will then submit the application on behalf of the member.

It will be understood that the present invention can be accessed via a text message as described above or through other means, such as through a proprietary mobile application, which an applicant can download and utilize by itself and which will act in a way similar to the text message method described above. In such a method, a member will login to the mobile application and then be directed to proceed through a similar process, resulting in the mobile application submitting the redetermination application for the applicant. It will be understood that the mobile application can create a more efficient process, removing the need for the user of the present invention to find the applicant, saving time and enabling MCOs to provide redetermination more efficiently. The mobile application aspect of the invention will also provide an MCO with added demographic information, filled in by the applicant through the mobile app., such as address, telephone number, desired language for communication, and gender, providing information about the member that the MCO did not previously have or could not otherwise compile.

The present invention utilizes tools, such as software designed to integrate a member's healthcare information into a database, so that an MCO can identify the acute and less acute healthcare needs of each of its members.

### BRIEF DESCRIPTION OF THE DRAWINGS

FIG. 1 is a schematic representation of devices used to implement the present invention.

FIG. 2 is a schematic representation of devices used to implement the present invention.

FIG. 3 is a schematic representation of devices used to implement the present invention.

FIG. 4A is a chart of acknowledgement questions of the present invention.

FIG. 4B is a chart of dependent questions of the present invention.

FIG. 4C is a chart of household asset questions of the present invention.

FIG. 4D is a chart of user questions of the present invention.

### DETAILED DESCRIPTION OF THE ILLUSTRATIVE EMBODIMENT

While the present invention is susceptible of embodiment in various forms, there is shown in the drawings a number of presently preferred embodiments that are discussed in greater detail hereafter. I should be understood that the present disclosure is to be considered as an exemplification of the present invention, and is not intended to limit the invention to the specific embodiments illustrated. It should be further understood that the title of this section of this application (“Detailed Description of an Illustrative Embodiment”) relates to a requirement of the United States Patent Office, and should not be found to limit the subject matter disclosed herein.

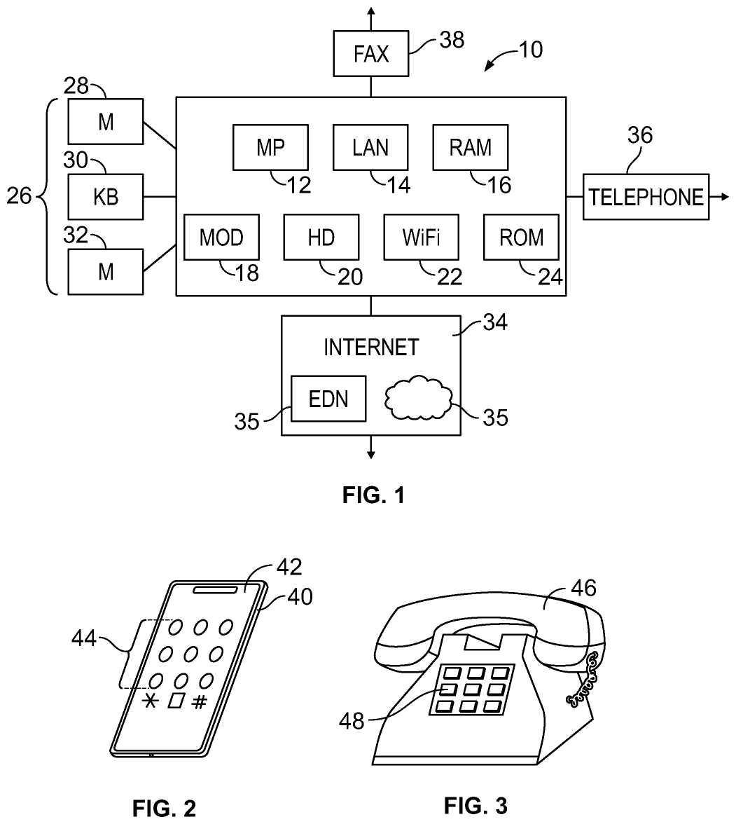

Referring to FIG. 1, a schematic representation of a computer 10 is shown, it will be understood that computer 10 can include all of the fundamental elements of a modern computer, and at least comprises the following elements: a microprocessor 12, a local area network (LAN) port 14, and associated equipment and software, rapid access memory (RAM) 16, a modem 18, a hard drive 20 for data storage, a wireless fidelity (Wi-Fi) adapter 22 and read only memory (ROM) 24. Additionally, the computer can be provided with input output devices 26, such as a monitor 28, a keyboard 30 and a mouse 32; such elements as bar code readers and camera can be included as well without departing from the novel scope of the present invention. Additionally, it will be seen that the computer 10 will have access to the Internet 34, to telephone transmissibility 36 and can include a facsimile device 38 for additional communications. Further, within access to the Internet 34, the user will have access to such things as cloud storage and enterprise data warehouses (EDW) 35, within which to store data.

Referring to FIG. 2, a mobile telephone 40, of the type having a touch sensitive screen 42 and comprising software and applications (Apps.) as well as standard touch tone communications means 44 is shown. FIG. 3, shows a standard telephone 46 having a touch tone keypad 48. As will be discussed in greater detail below, the devices of FIGS. 1 through 3, or their equivalents, are instrumental in the means and method of operating and using the present invention. Persons having ordinary skill in the art will understand that the representations in FIGS. 1-3 are generically made and that actual computers, mobile telephones (and other devices, such as pads) and standard telephone of various manufacturers, manufacture and design can be utilized without departing from the novel scope of the present invention.

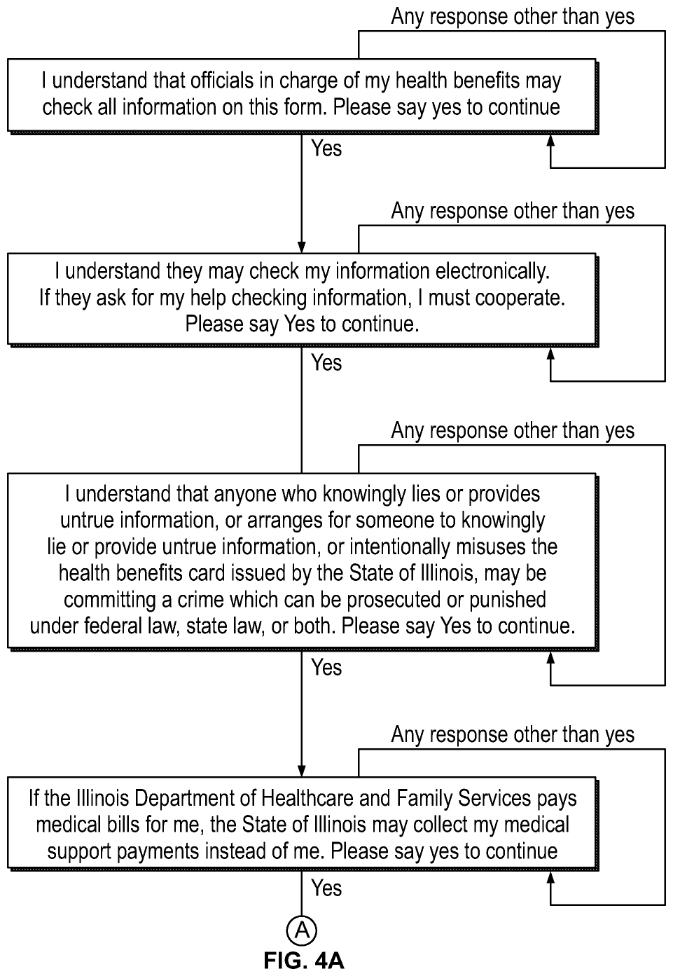

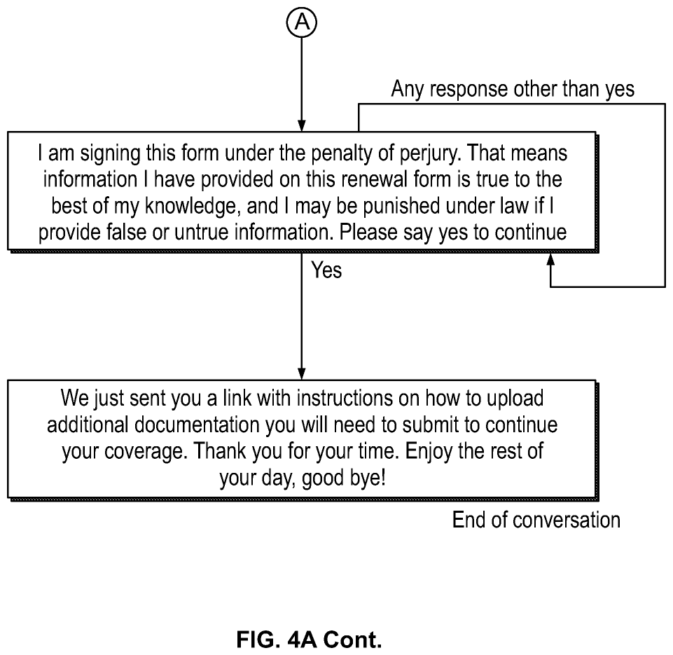

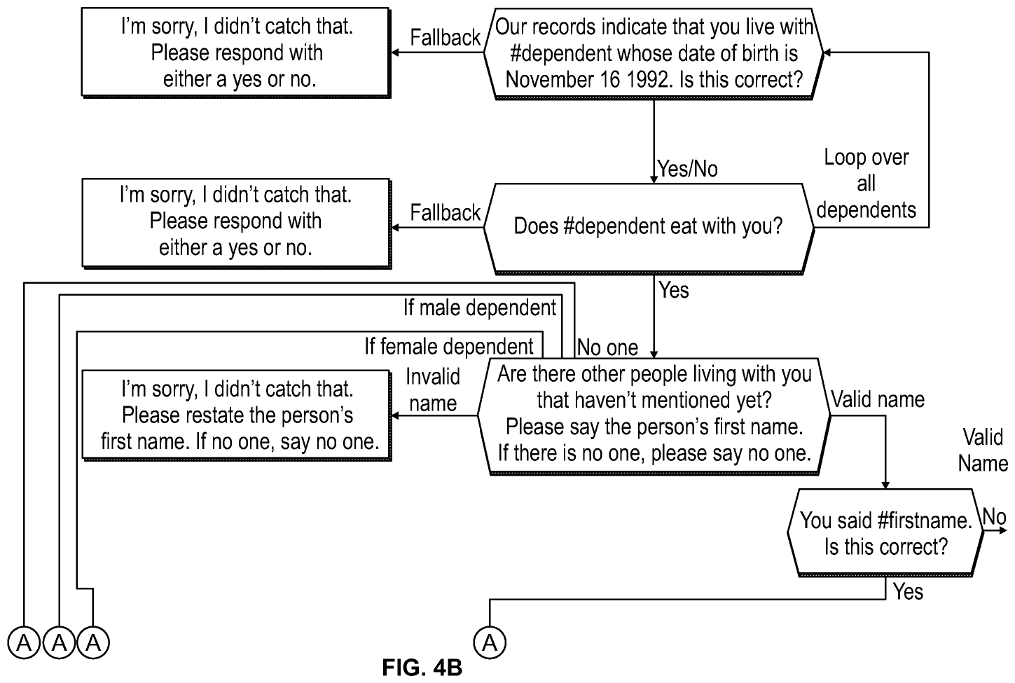

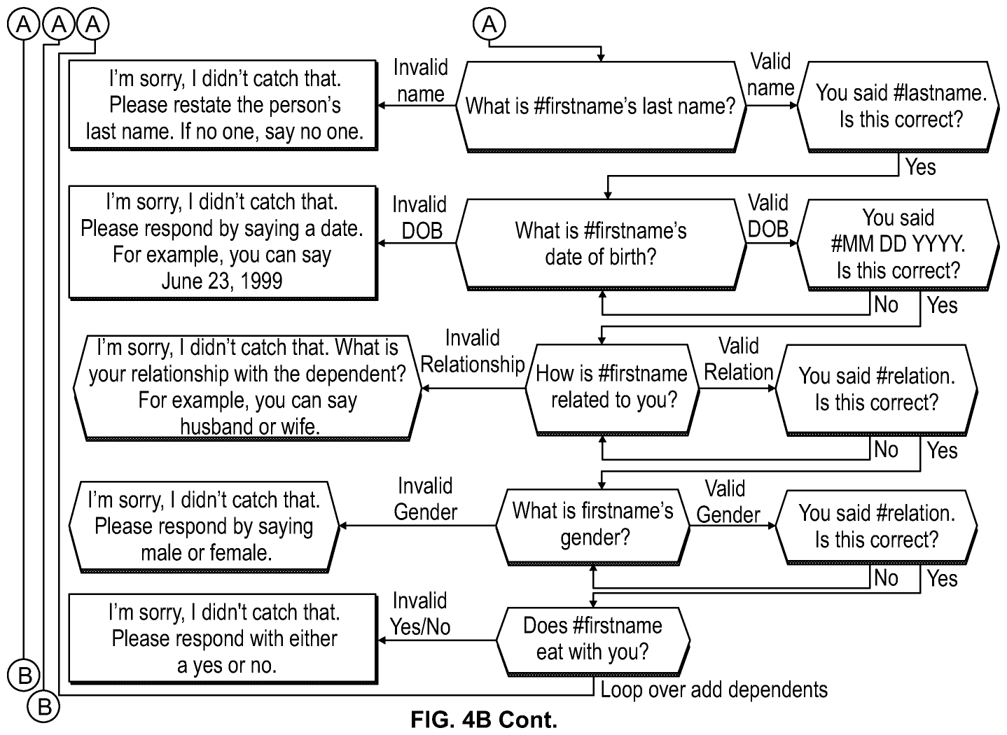

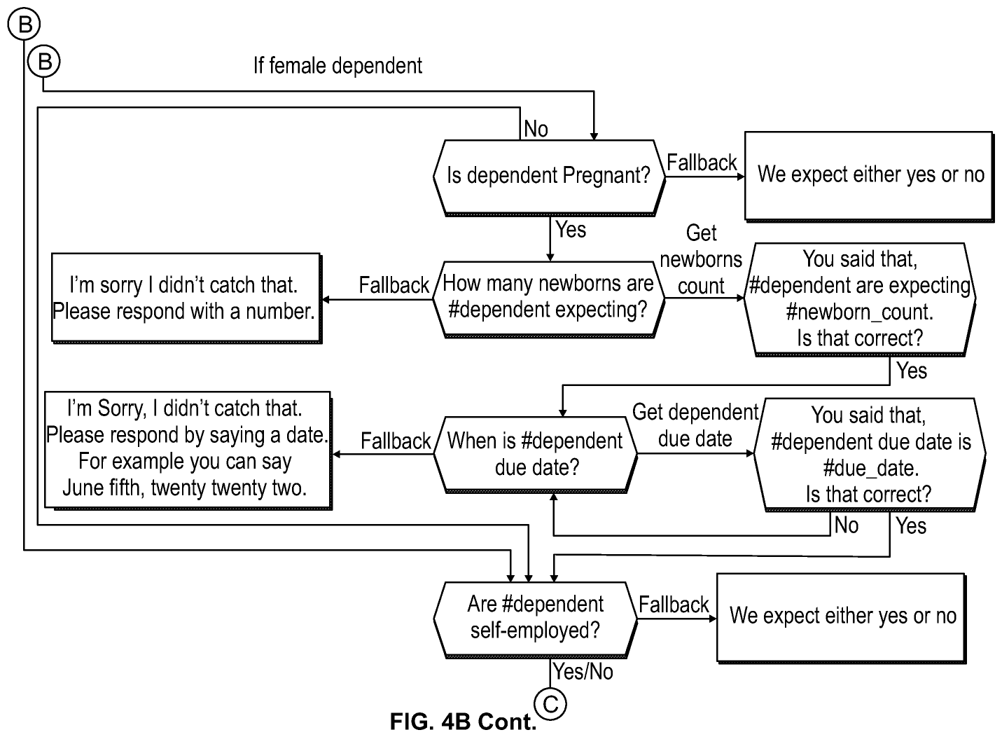

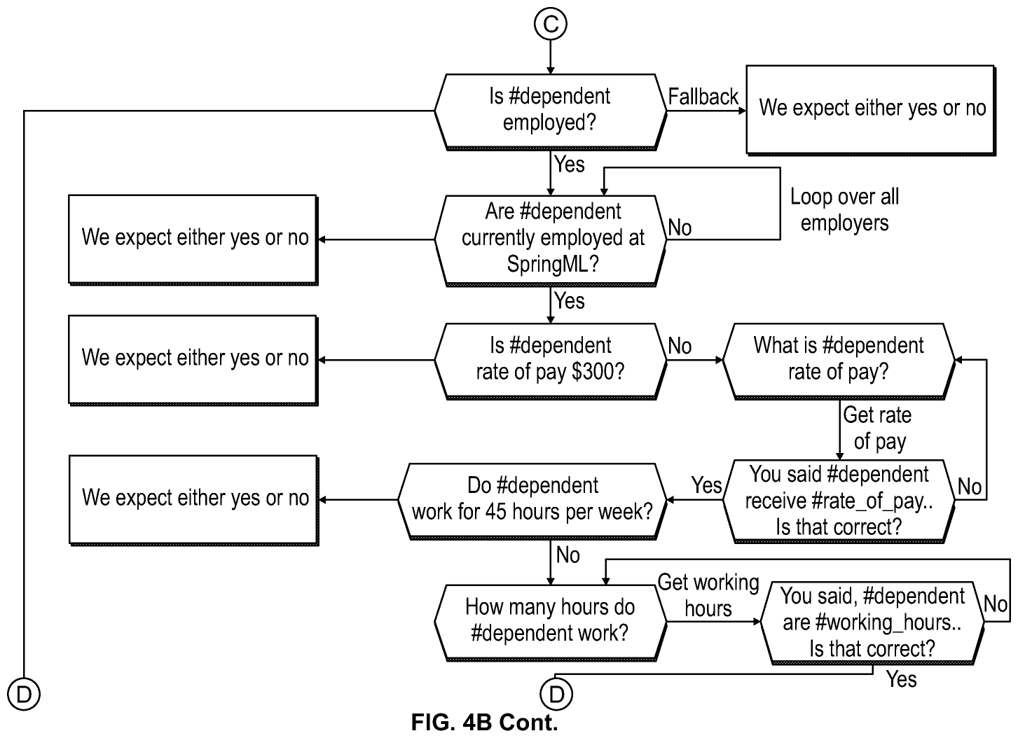

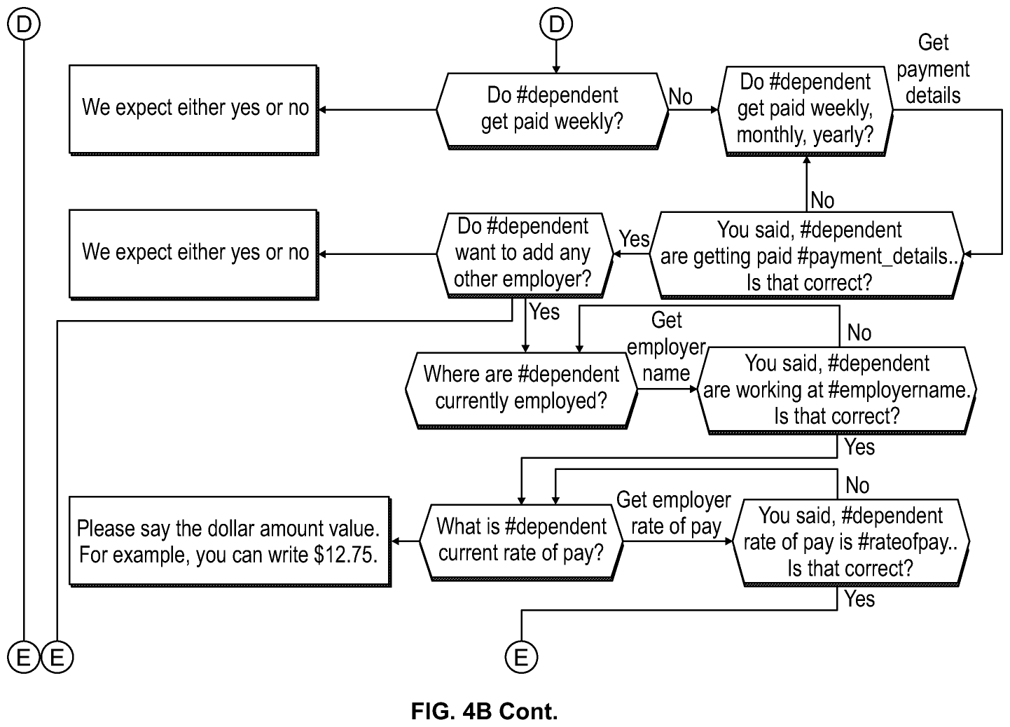

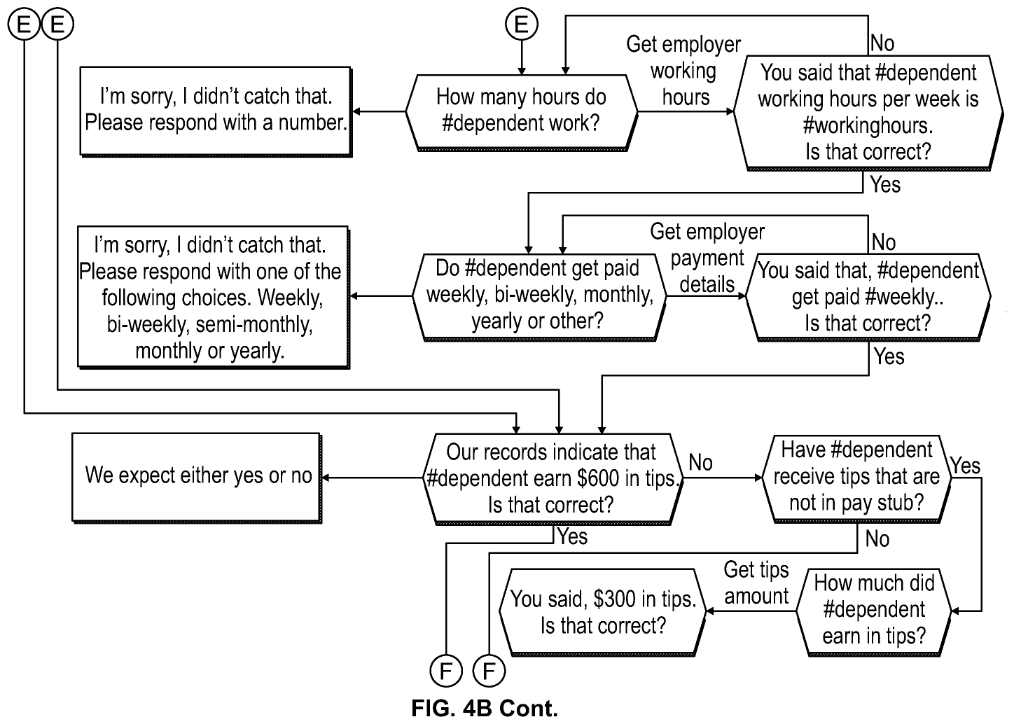

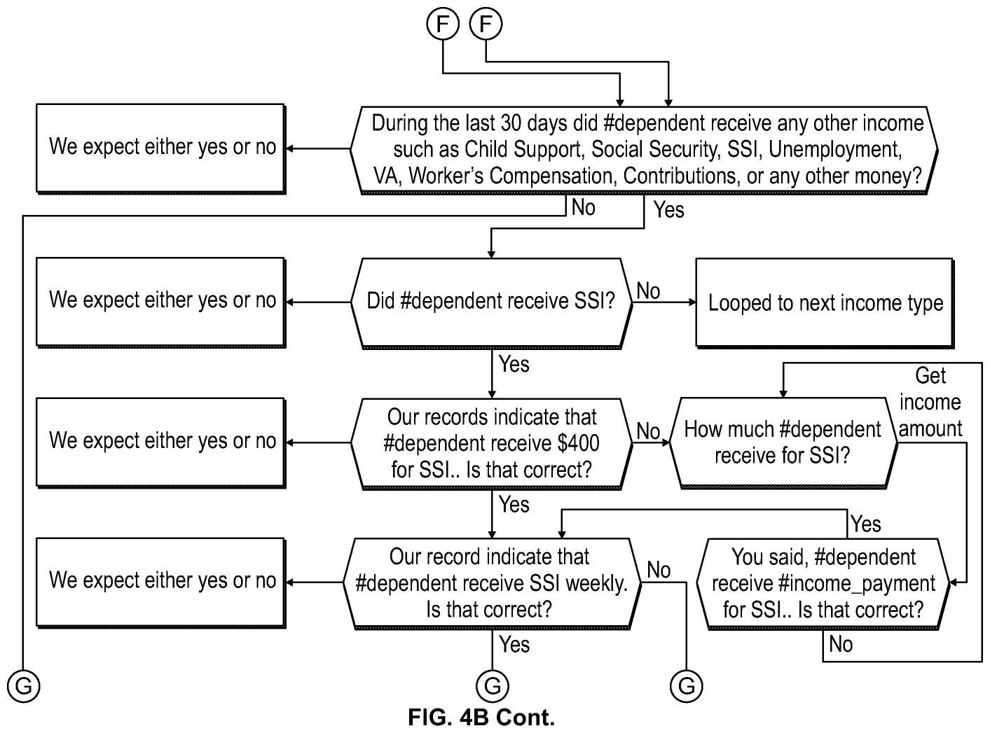

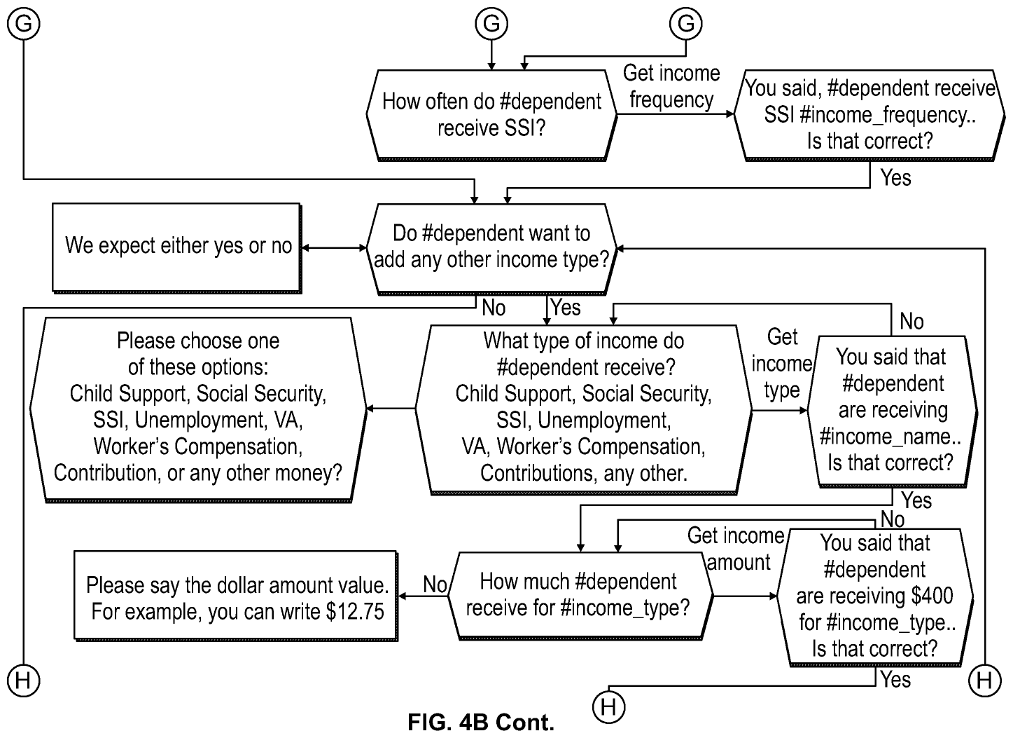

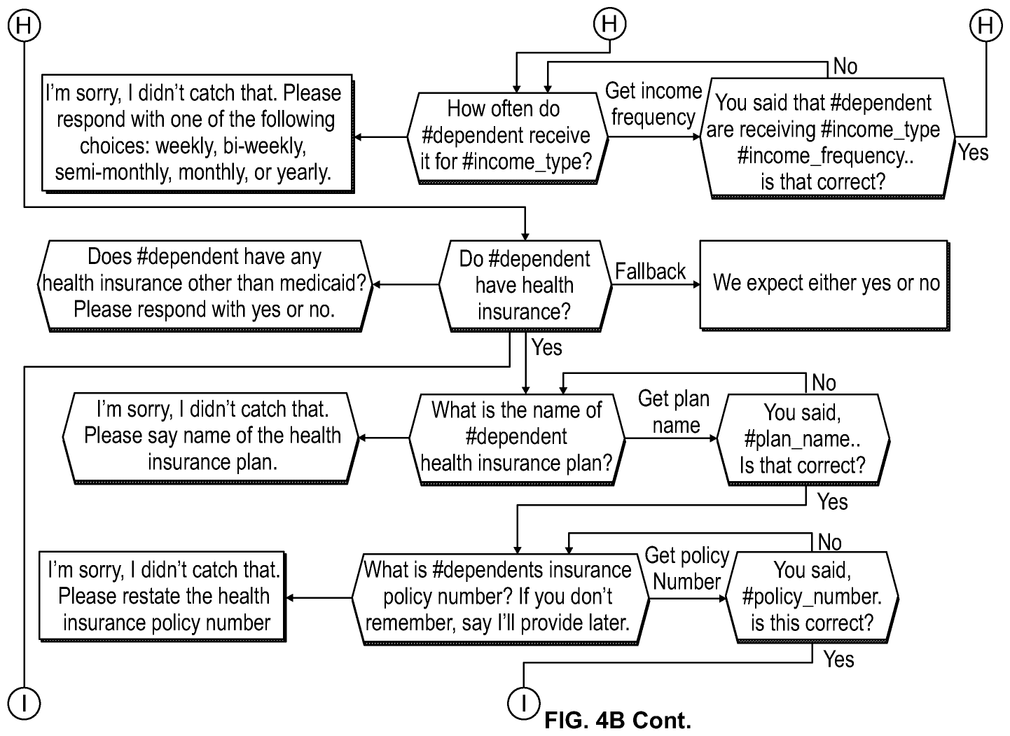

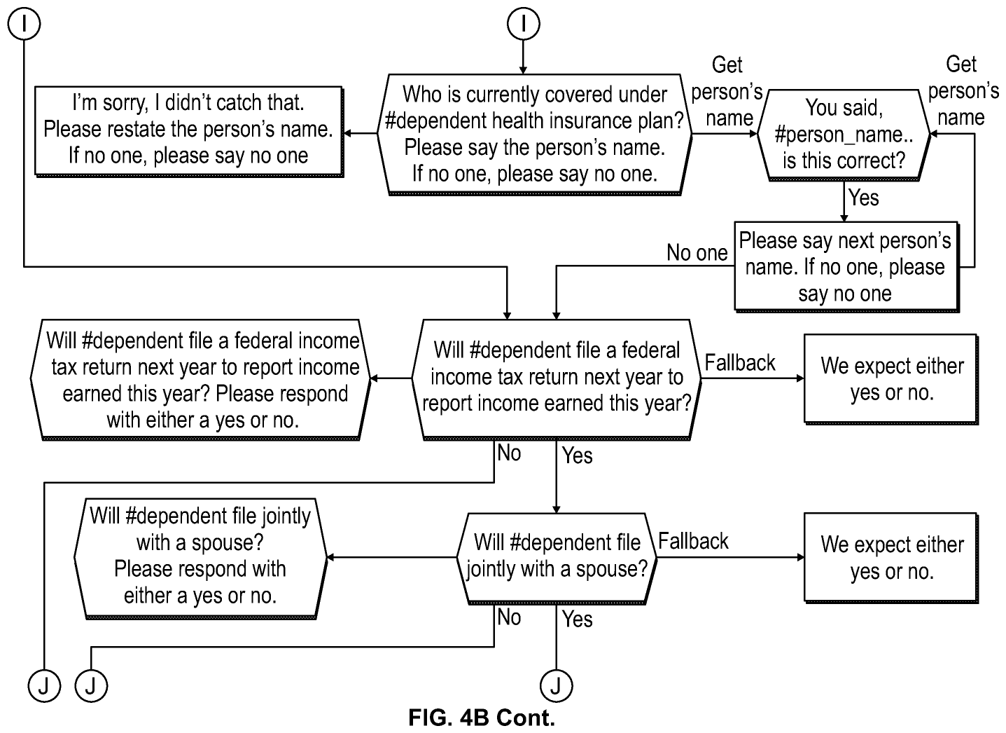

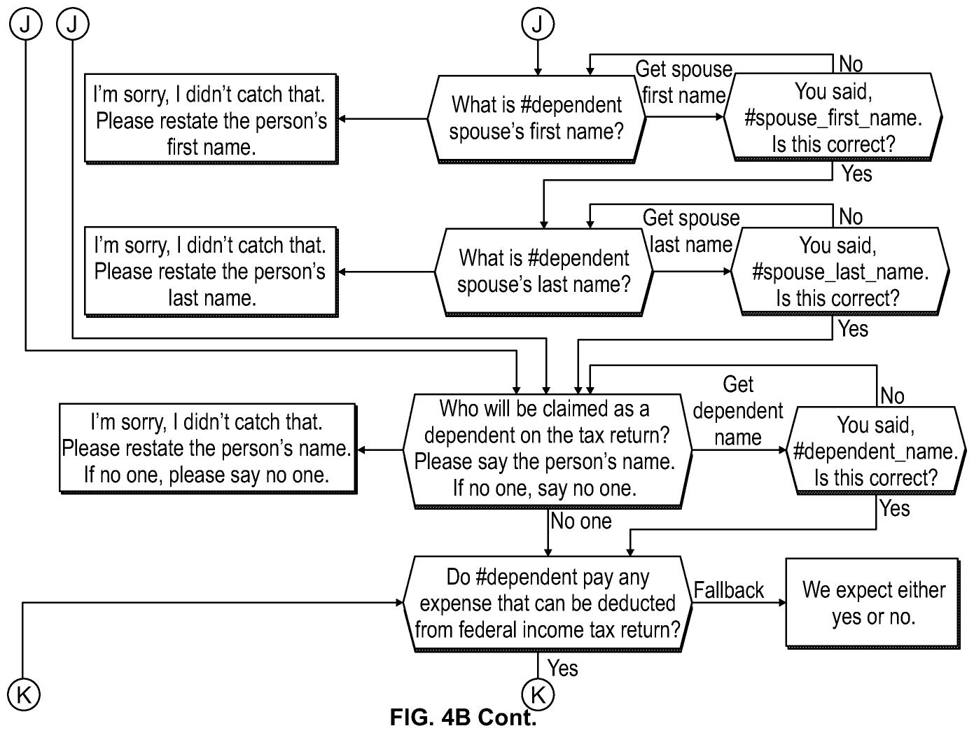

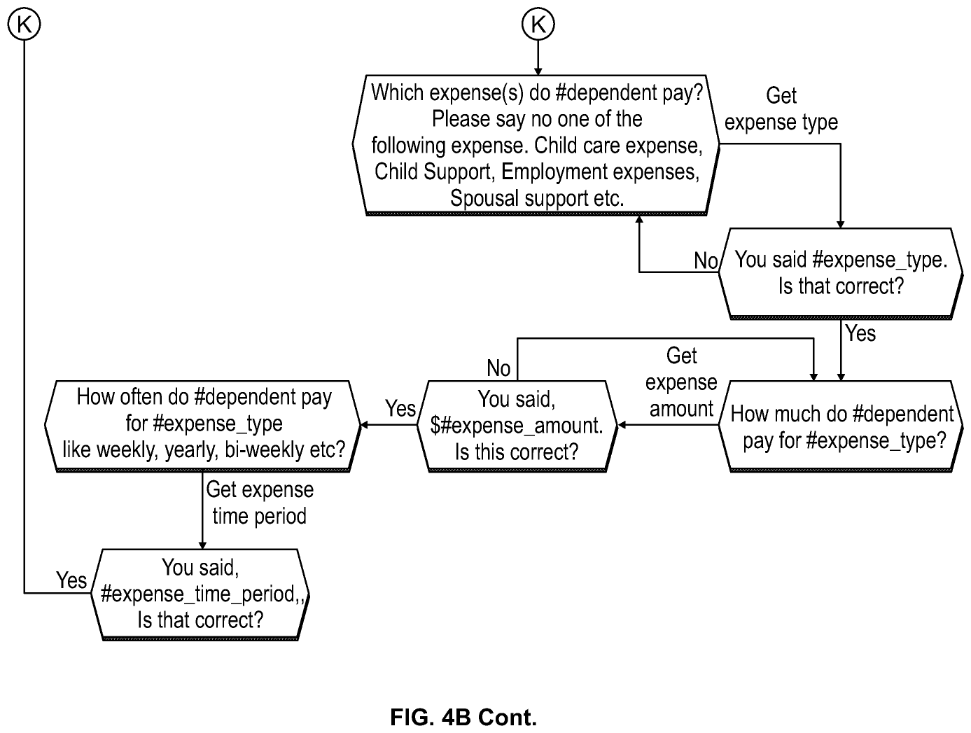

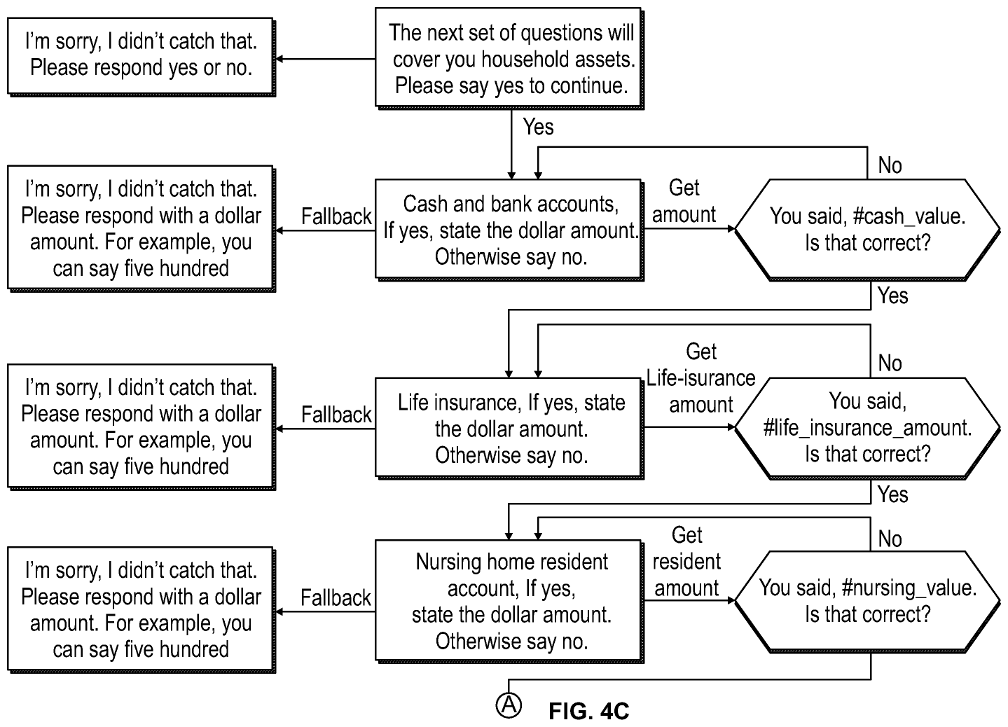

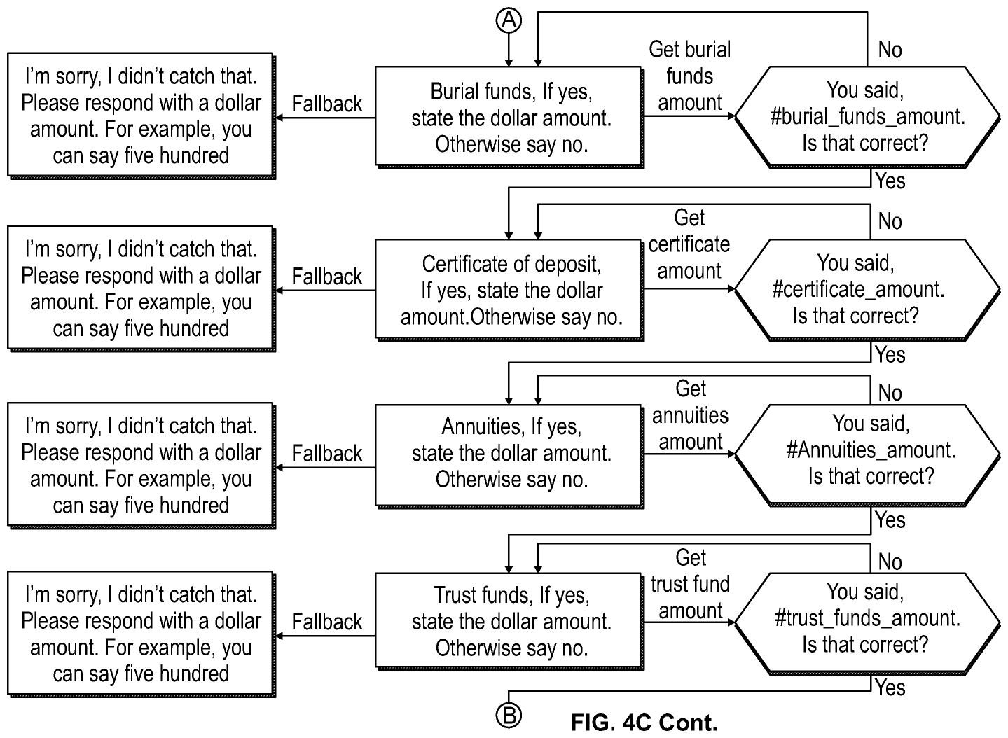

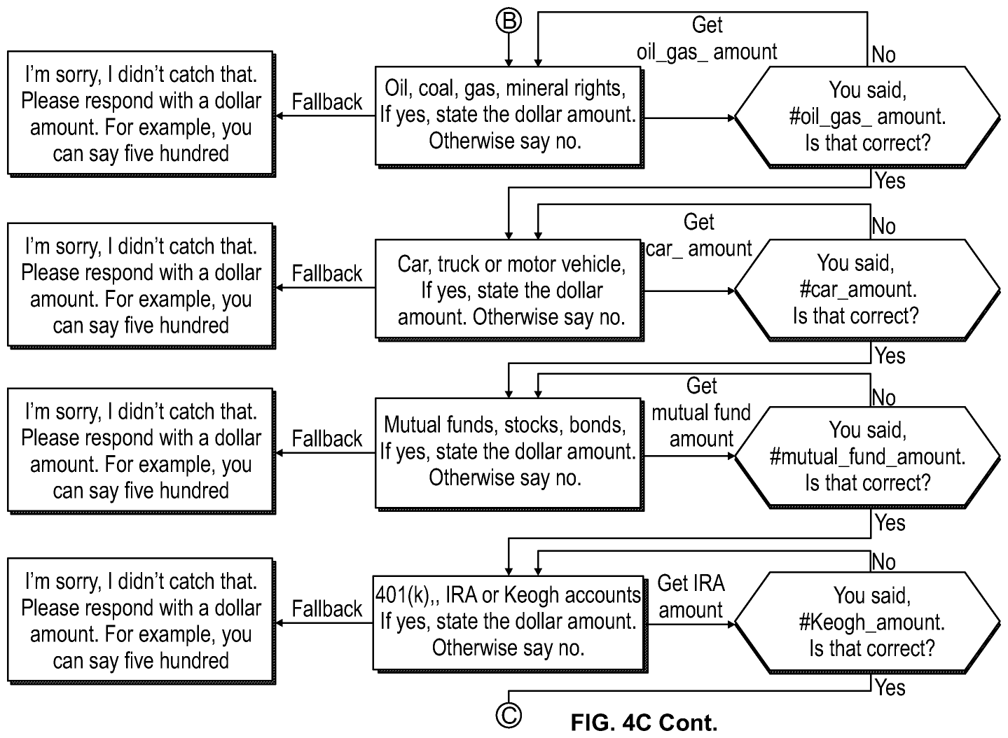

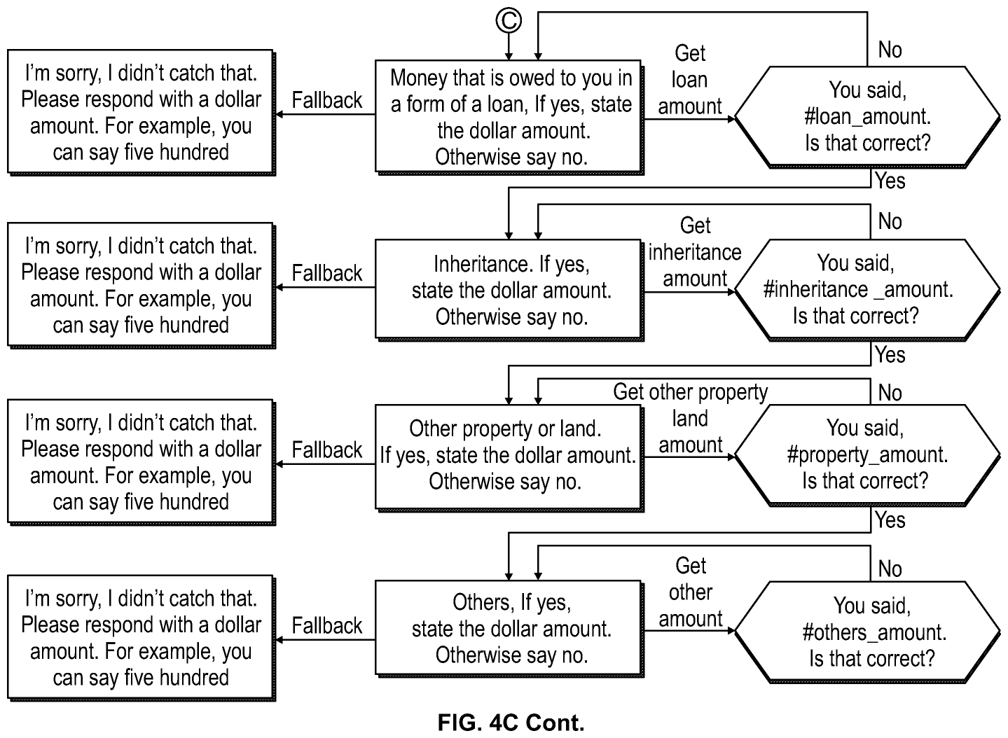

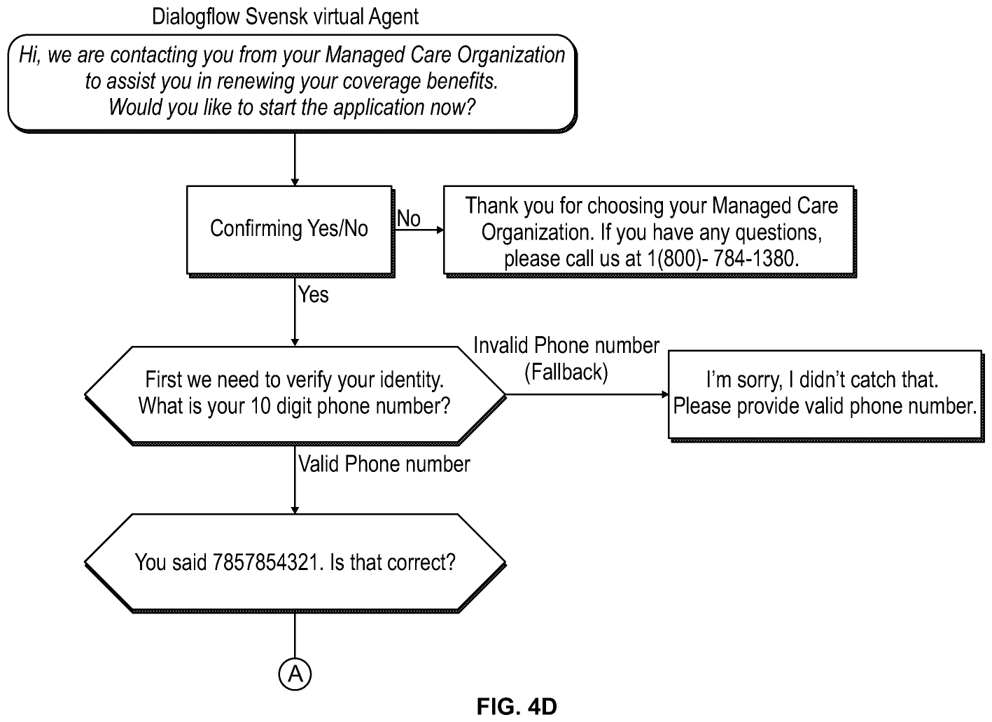

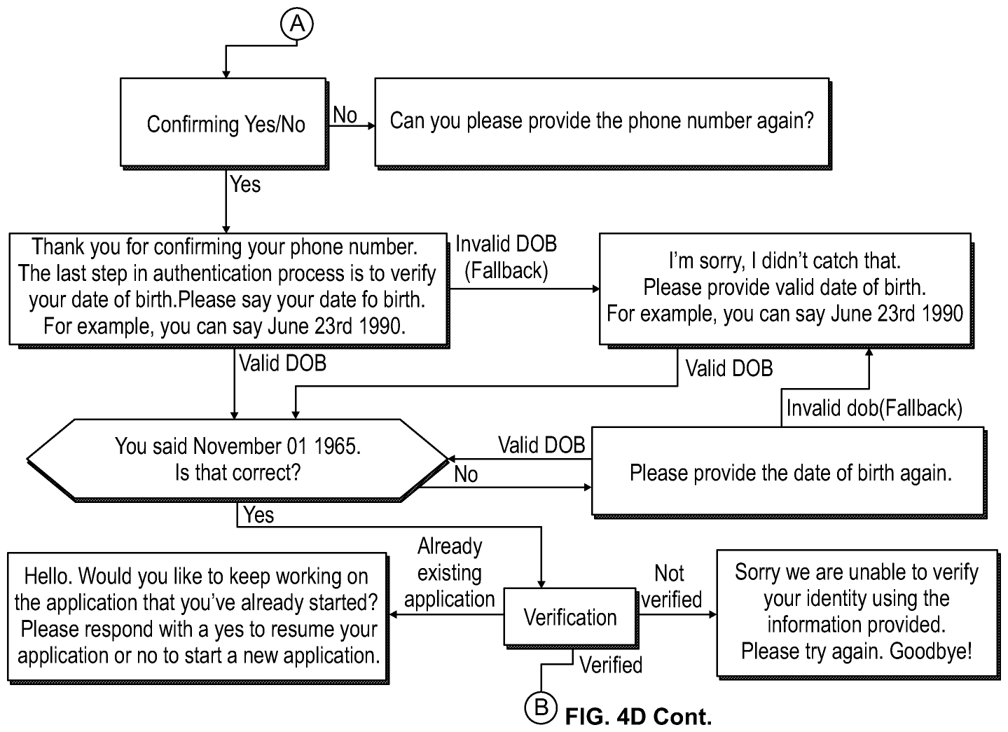

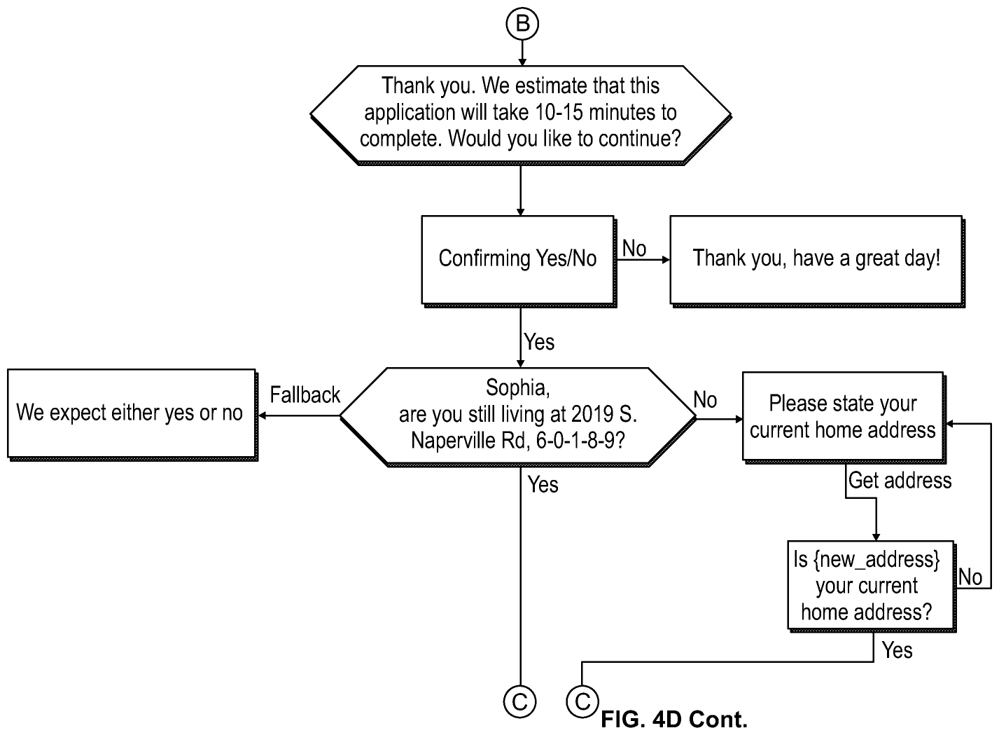

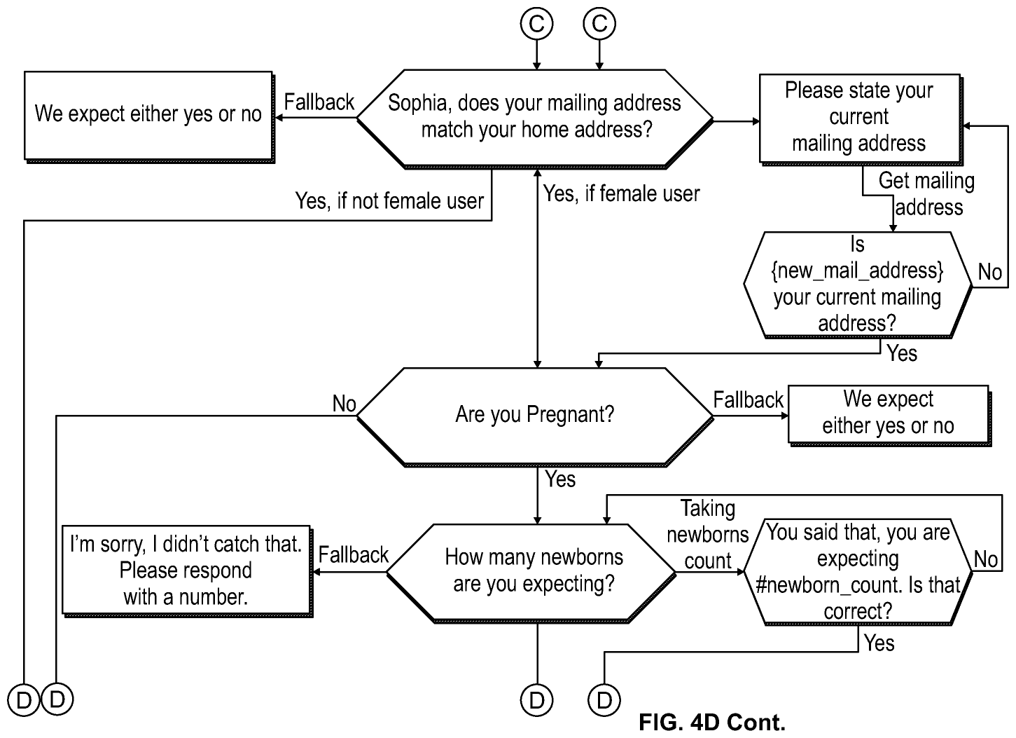

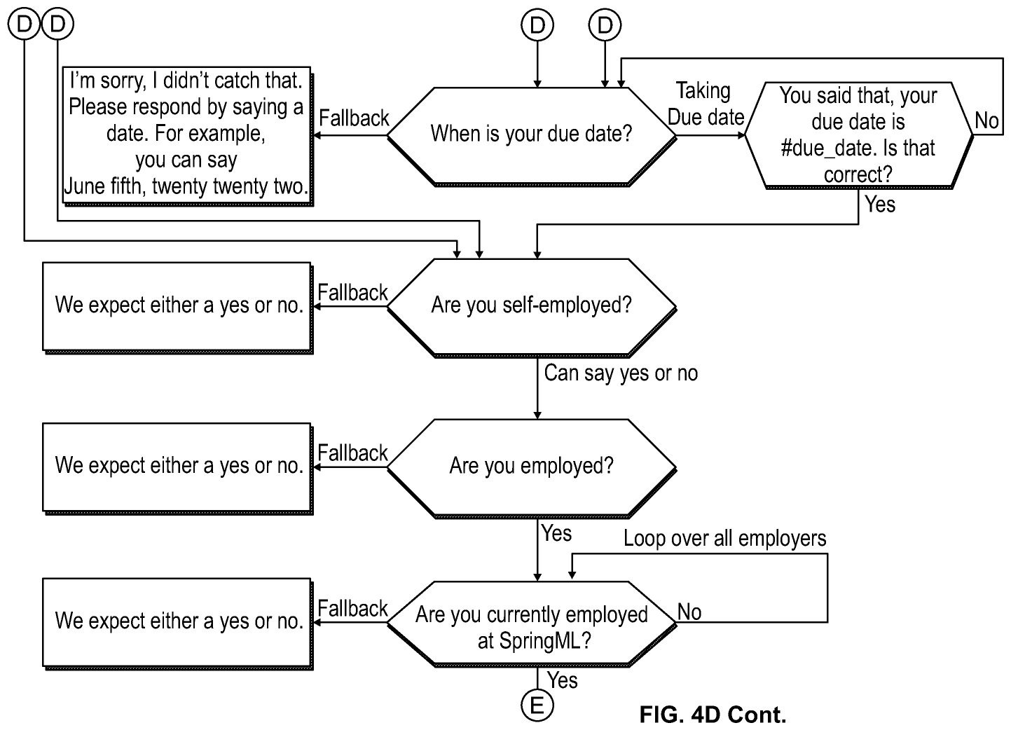

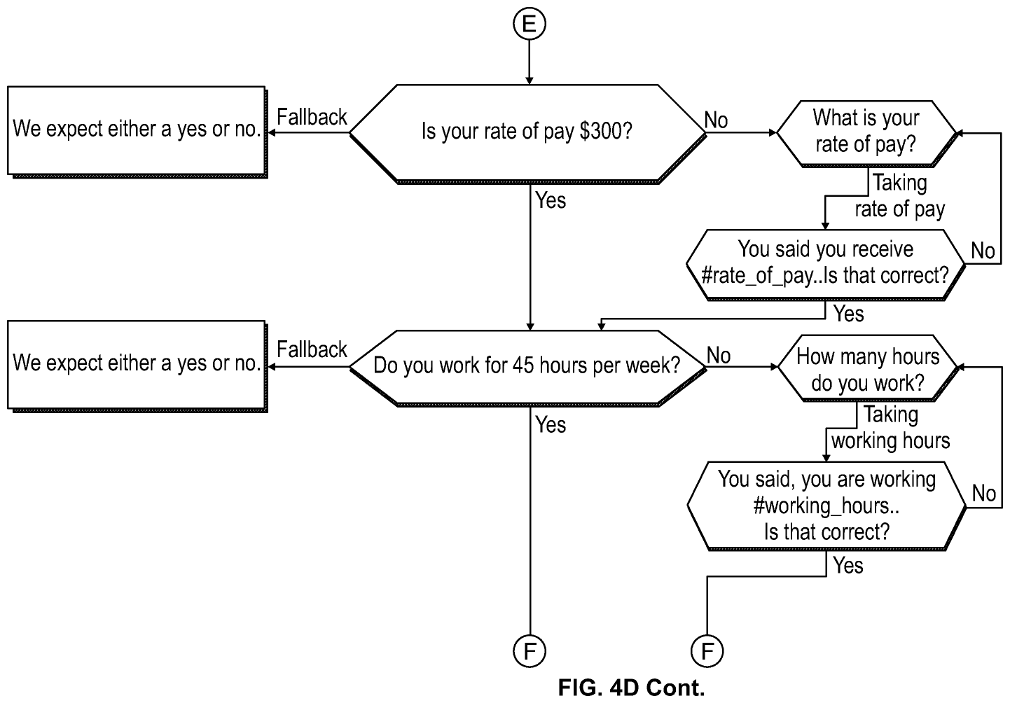

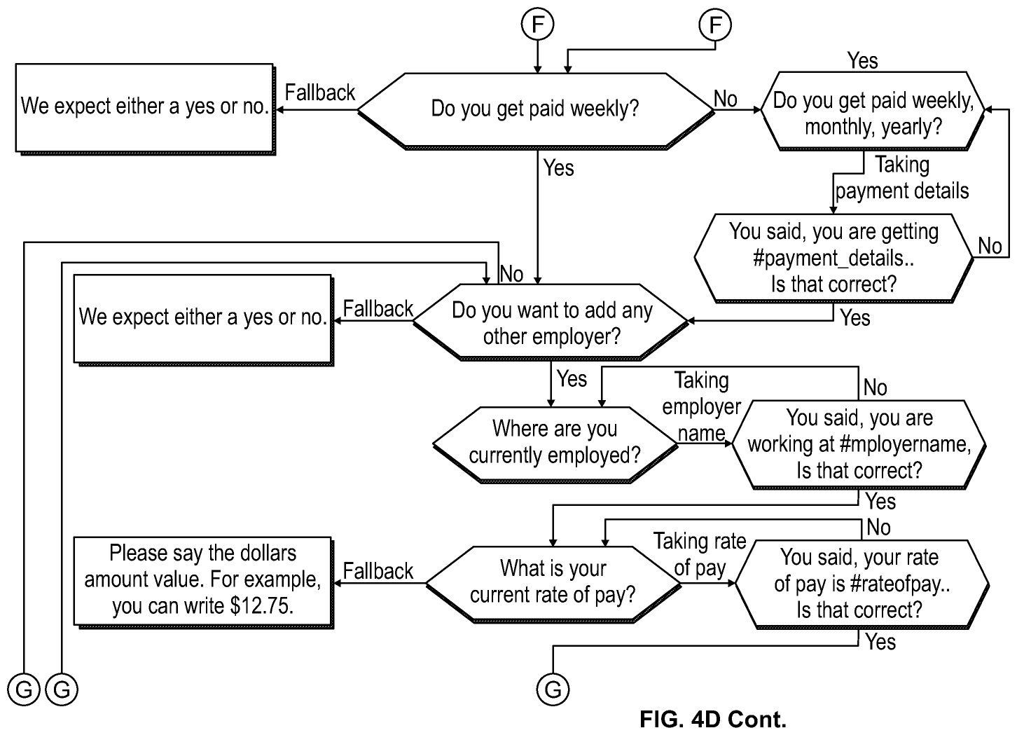

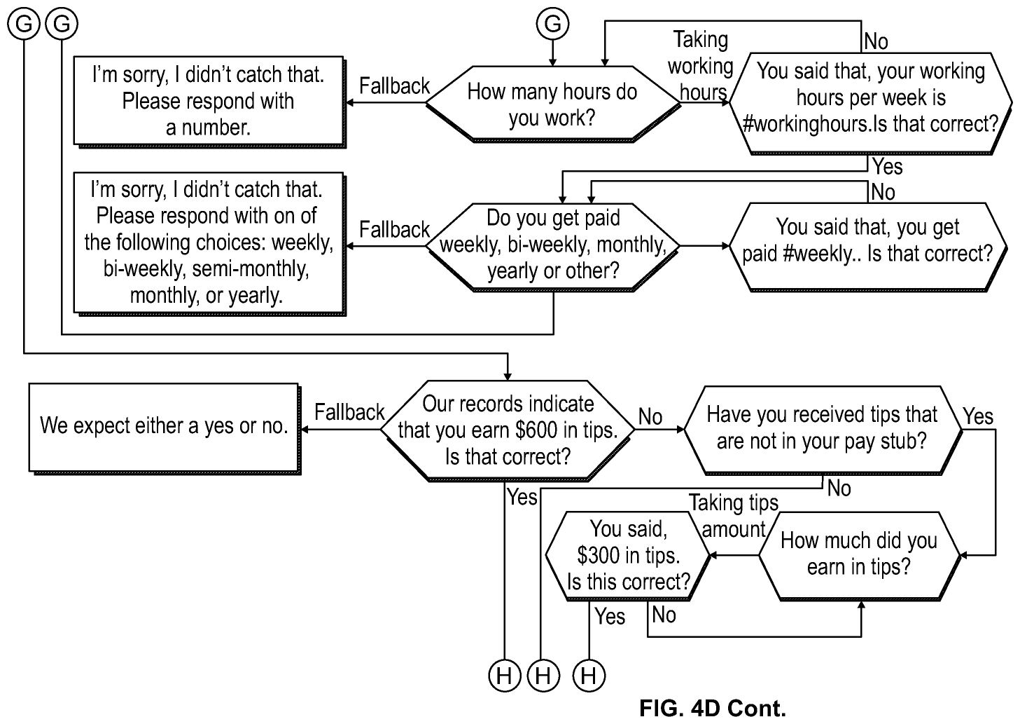

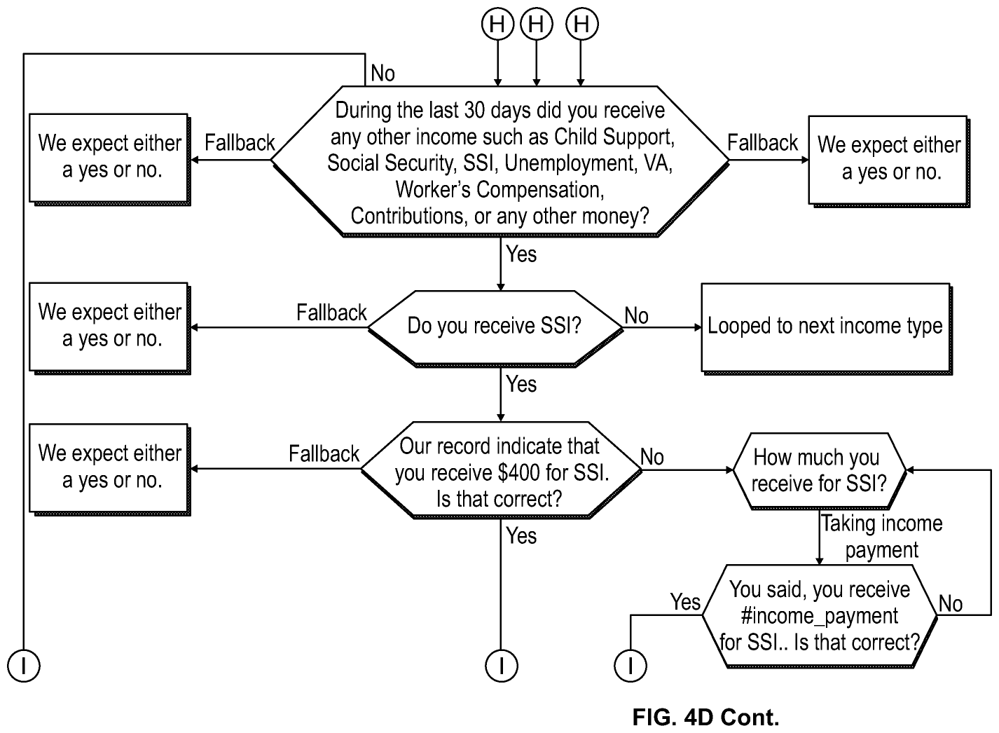

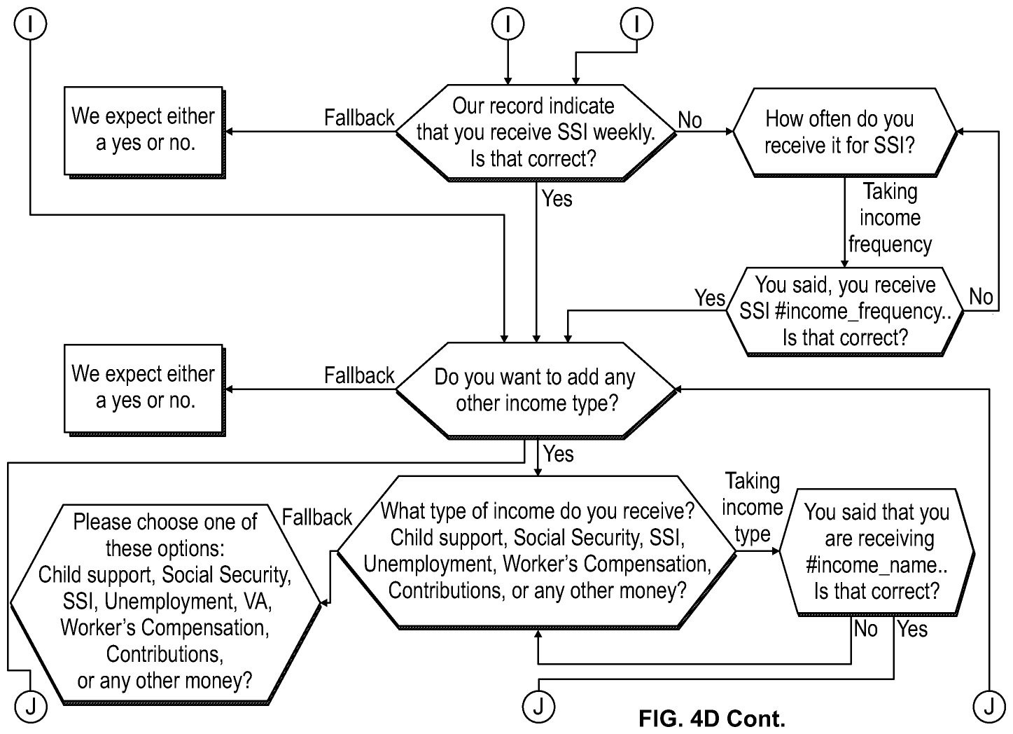

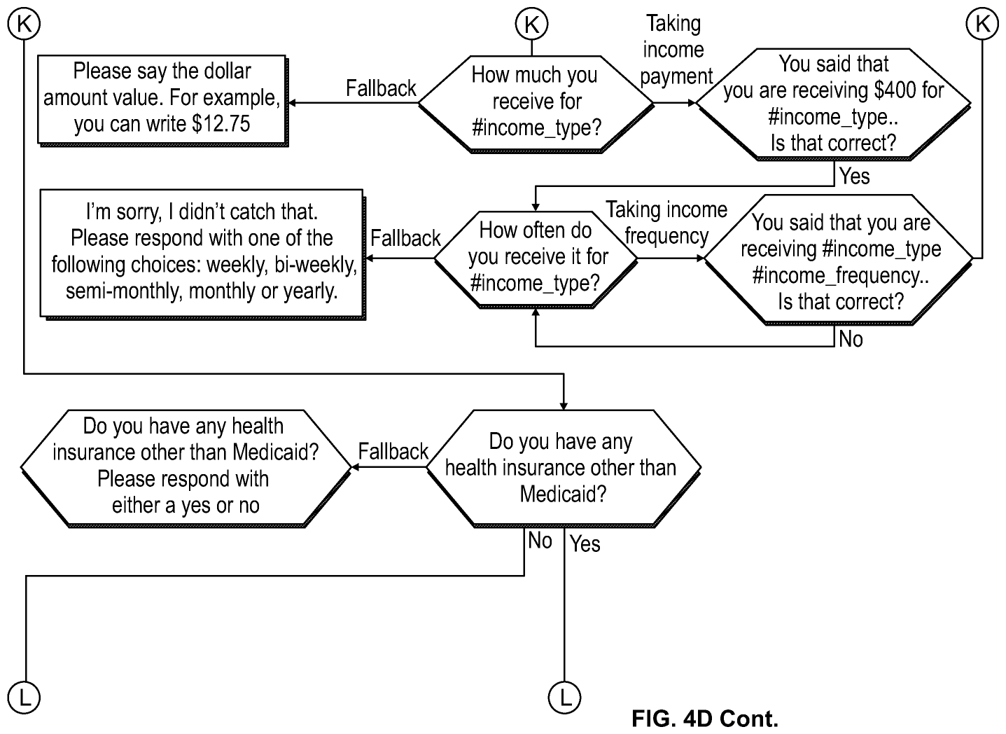

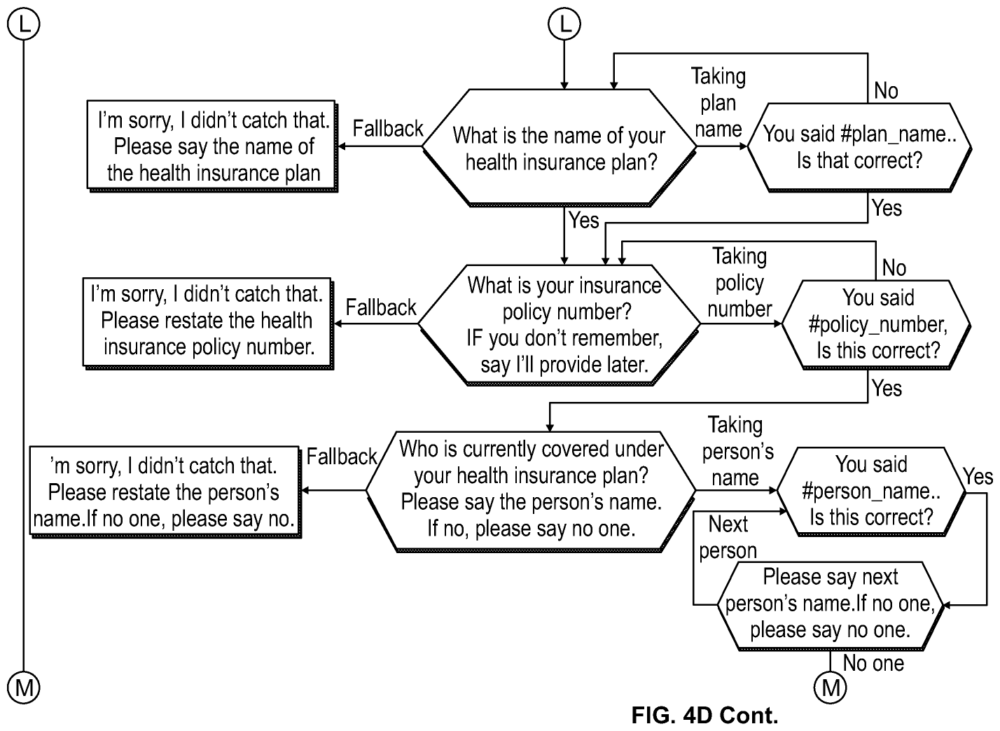

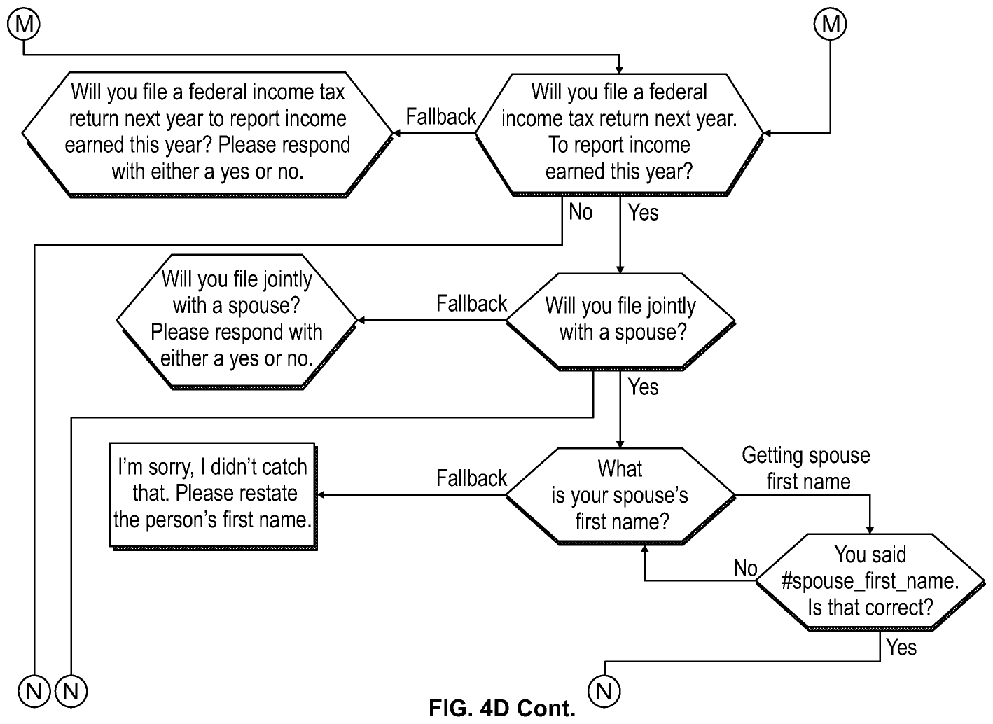

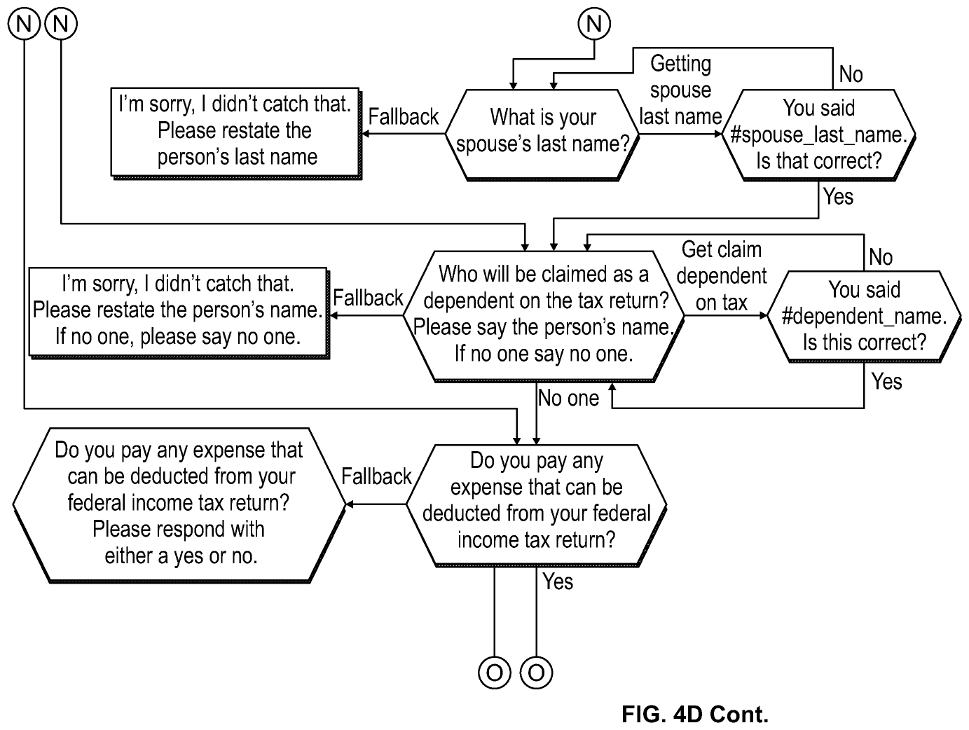

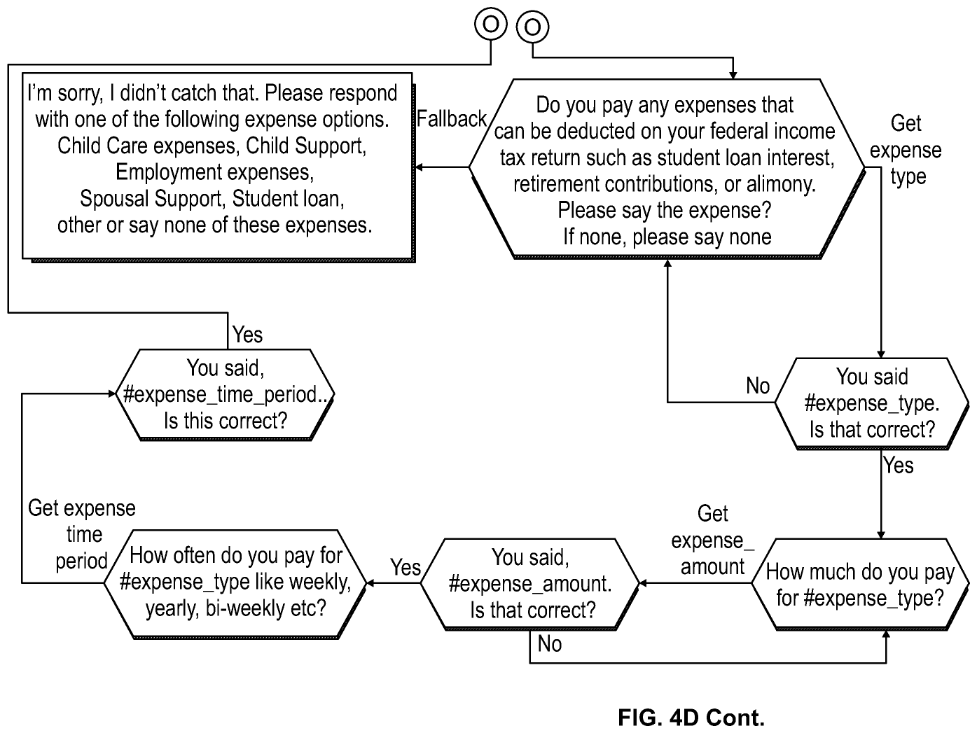

Reference is now made to the Redetermination Question Sheets, shown in FIGS. 4A, 4B, 4C and 4D. It should be appreciated that FIGS. 4A, 4B, 4C and 4D represent lists of questions and answers, in the form of a flow, such as an interview that can be conducted over a telephone call or through computer or mobile device application where persons are questioned (either through written questions or questions read to them via their computer or mobile app) that when asked will determine eligibility and also direct the applicant/member to the form(s) necessary for determination or redetermination in a preferred embodiment of the present invention, but that other questions and answers can be introduced/inserted, within the lists here, or the lists can be designed differently to achieve the desired result. Substitutions of questions and answers, as needed, can be made, without departing from the novel scope of the present invention. The answers to the questions determine the appropriate forms to use, the jurisdiction of the applicant, eligibility to participate and provide information for the automatic filling in of forms necessary to participate in the program.

A flow description of the process for redetermination is provided below.

### Redetermination Process Flows as the Following Steps

### Step:

1. A Managed Care Organization (MCO) will pull the member's name and any information they have on the member 60 days prior to the redetermination due date and then again 30 days prior. The MCO will develop a file extract utilizing the data from the 834 monthly enrollment file, which is the electronic HIPAA transaction that MCO's retrieve monthly from the State Department that manages the Medicaid program, for example, the Illinois Department of Health and Family Services (IHFS), that reflects its members for the following calendar month. In addition, the MCO will pull and send claims data, to include medical, behavioral health, and pharmacy.
2. A cloud storage provider, referenced on the chart as EDW (Enterprise Data Warehouse) 35 (in FIG. 1) such as Google or other data handler that is “SOC 2” certified¹, will be engaged to manage all the provided or created information subject to the Health Insurance Portability and Accountability Act of 1996 (HIPAA). The extract file information that includes the 834² monthly enrollment data will then be sent to the storage provider from the MCO. The MCO sends the enrollment file that includes, at least, the member's Medicaid ID Number, telephone number and address (if applicable), redetermination date, Date of Birth, language (although very limited). It will be understood that, for example, the Illinois Department of Healthcare and Family Services (HFS) has published a data dictionary, on its own website, that includes most of the data elements included in the 834-enrollment file and it is expected that many or all of the other U.S. states have similar publications, the Illinois data dictionary being used, herein, as an example of such publications. ¹SOC 2 is a voluntary compliance standard for service organizations, developed by the American Institute of CPAs (AICPA), which specifies how organizations should manage customer data. The standard is based on the following Trust Services Criteria: security, availability, processing integrity, confidentiality, privacy.²The ANSI 834 EDI Enrollment Implementation Format is a standard file format in the United States for electronically exchanging health plan enrollment data between employers and health insurance carriers.
3. Once the data is collected and saved, the data will then be pulled from the secure data room by proprietary technology of the present invention, which will initiate the break down and analysis of the data thus far collected, then perform data mining, and through an analytical process identify and find the individuals for redetermination or recertification.
4. One of the key components of the present invention is the data mining step—which uses the technology to do the data mining of the data that is received to identify members who might qualify for Supplemental Security Income (SSI) and/or Social Security Disability (SSDI).
5. The next step in the process is the breakdown and categorization of the members into two categories—a list of members who may qualify for SSI or SSDI and redetermination and a list of members due for redeterminations and Health Risk Screen (not SSI and/or SSDI eligible).
6. If the process selects the use of mobile telephone communication technology, the process enables the MCO to send out a “welcome” text message to the member. The text message, in a preferred embodiment, will include an opt-out option. If the member does choose the opt-out option, then the member will be directed to a call center to assist that member with questions and/or with the redetermination process.
7. If the member responds with an opt-out, then they will be automatically redirected to a human at a contracted and secure call center. This part of the process will be further explained in step 10 below.
8. If, through the data mining step, it is determined that the member's telephone number does not work, then the process will utilize the current technology to perform a skip tracing function to try to locate the member. This step will include the use of available online and public information and data systems such as those offered through a number of different vendors, such as LexisNexis and Smart Dial technology, to perform a search in an attempt to find an active telephone number as well as other personal information for the member.
9. Then the next step in the process is for the above acquired data to be sent to the cloud provider or data handler by appropriate technology, as will be understood by persons having ordinary skill in the art. The member file can then be retrieved by the MCO to make further attempts to reach and notify the member of the need for determination or redetermination of their status within Medicaid. This data can include updated members' demographics from a Health Risk Screen and/or Skip Tracing. The Health Risk Screening is a screening tool administered by MCOs to all new Enrollees and provided within sixty (60) days after enrollment, to collect information about the Enrollee's physical, psychological, and social health.
10. If the member responds, and does not opt out, then the technology of the current method will automatically send out another text that will provide the necessary details about the redetermination process while also then directing the recipient to the appropriate (usually state government website). In addition, the technology of the current method may also send out regular mail (postal service) to members that do not proceed with the process in yet a further effort to track down and help guide the member to complete the necessary paperwork for the redetermination process.
11. After this process is completed, the present invention will send a file, with all the known and updated data, to the cloud storage provider (such as Google® or other storage provider or data handler) using the equipment shown in FIGS. 1-3. This data can include key information such as the member's demographics, heat map, population engaged/age/sex etc. The MCO can then retrieve this file from the cloud storage provider (or other storage provider or data handler) using the equipment in FIGS. 1-4, as known by persons having ordinary skill in the art.

As part of the overall system, which is a key component of the invention, is the member's ability to go from using a virtual agent or voice chat initiated by the text message sent in Step 6 above to an online web form with a virtual text chatting feature to assist along the way and even allow the member to return to a real human assistant seamlessly and without losing any data input. A particular unique feature of this invention is that it allows the member to start the process through one of the means available, stop at some point and then reconvene using the same means or transferring the process to another means available through the system without losing any data or input. Unique to this system is that the member can switch from using a virtual agent (using artificial intelligence) on the telephone, to an online web form with a virtual text chat assistant to an old-school human assistant. Whether the member uses the virtual assistant, online form or a human assistant, the process is made easy by the system which walks the member through the data input using a series of easy-to-understand question and inquiries. Of course, another important feature of this invention is that any of these data collection input means can be performed in any language to assist members who do not use English as their main source of language. It should be further understood that the initial text message will allow for the entire redetermination process to be performed and processed over the mobile telephone or the member can click on a web link that is sent to the member if they prefer working online through the website form. A sample of the online forms, used to inquire and gather data, are included herewith in FIGS. 4.

What the member will also find is that in many cases the redetermination form is already pre-populated with the member's then known information, which of course can be uploaded during the redetermination process as the member is walked through the data fields in order to confirm or update the member's information, such as address, employment. This cuts down on the member's time in filling out the form, as in many cases 50% or more of the member's data is already available and pre-populated. In many cases, the ordinary, or average individual, can complete the redetermination process in about 15 minutes. This greatly increases the percentage of persons that will take the time necessary to be redetermined.

Other features of the present invention is that the system will send reminder text messages to members that may have started the process but for whatever reason did not complete the redetermination process. The system does not only send reminder and follow-up messages but will also flag errors or data points that may still need to be completed or confirmed in order to complete the redetermination process.

Further, the inventive system also provides for confirmation and verification of the member being sent the initial redetermination text message so that confidential personal information is maintain in secret to the highest desired or required level. Part of the confirmation process, but by no means the only possible means, is with a two-step verification of telephone number and birthdate.

This inventive redetermination process, including all the input/intake means are all ADA compliant. Further the process can include, as a step in the process, means for providing the member, who has successfully completed the redetermination process, a financial reward. Such a reward can include a gift card that can be used by that member. The amount of the gift card can be conditioned on the responsiveness of the member or dependent on other factors such as age, living conditions, abode as well as other factors or criteria. Of course, the member is advised of this reward at the inception of the redetermination process and all along the process as the member inputs and/or updates their information regardless of the means (telephone, mobile app, online, human assisted) being used by that member.

While the present invention is capable of embodiment in various forms, there is described herein the presently preferred and some known alternative embodiments. It should be understood that the present disclosure is to be considered as an exemplification of the present invention, and is not intended to limit the invention to the specific embodiments discussed or illustrated. Although an illustrative embodiment of the invention has been shown and described, it is to be understood that various modifications and substitutions may be made by those skilled in the art without departing from the novel spirit and scope of the invention.

What is claimed is:

1. A method of finding and enrolling or re-enrolling a person in a specific desirable program, using telephonic, computer and electronic communications means, comprising the steps of:
providing means for communication with a candidate or person assumed to meet requirements for admission to the desirable program and if communication is not made, automatically using a search program and computer to find personal contact information of the candidate to then reattempt communication therewith;
storing the personal contact information of the candidate in a secured rewritable and searchable computer database having a calendar;
utilizing the personal contact information to communicate with the candidate;
determining if the candidate is eligible for the desirable program, and if so, renaming the candidate an applicant;
directing the applicant to an appropriate enrollment means for providing information for enrollment in the desirable program;
securing enrollment data, through the enrollment means, from the applicant, and storing the enrollment data in the secured rewritable and searchable database, and associating the data with the personal contact information for the applicant as applicant data;
automatically, by allowing the computer to use the applicant data, merging the applicant data to a precise computer chosen application form to create an application for enrollment in the specific desirable program and submitting the application form for enrollment; and
updating the applicant data and associating the data with the calendar to automatically contact the applicant, using the updated applicant data, within a specified time period for re-enrollment.

2. The method of claim 1, wherein enrollment data can be secured via a telephone conversation, a questionnaire on a mobile device or a questionnaire on a personal computer.

3. The method of claim 2, wherein securing enrollment data can be started on any one of a telephone conversation, a questionnaire on a mobile device or a questionnaire on a personal computer and completed, later, on any one or more of a telephone conversation, a questionnaire on a mobile device or a questionnaire on a personal computer.

4. The method of claim 1, wherein the specific desirable program is Medicaid.

5. The method of claim 1, wherein a computer includes any electronic data entry and manipulation devices including one or more of a personal mobile device, a mobile telephone, an electronic pad, an electronic tablet, a personal computer, a laptop computer or any other data entry device and components thereof, including software and peripherals.

6. The method of claim 1, wherein enrollment data is created by the applicant answering a series of questions, the answers being automatically recorded in the secure database.

7. The method of claim 1, wherein when an attempt to communicate with a candidate is unsuccessful, the process automatically uses means to search public and private databases to find updated contact information and then uses the updated contact information to again attempt to communicate with the candidate; this process continuing until communication is made.

8. The method of claim 1, including the step of utilizing the means for communication and storage to periodically scour the regulations of the specific desirable program to allow for automated changes in one or more of the questionnaire, forms and enrollment data as required for enrollment as a result of changes to laws and/or regulations associated therewith.

9. The method of claim 7, where membership is limited to a particular jurisdiction and the process includes means to notify a provider in a second jurisdiction of a candidate who has moved to the second jurisdiction from the particular jurisdiction for enrollment of the member therein.

10. The method of claim 2, wherein the questionnaire includes a Health Risk Screening to help assess the candidate's present health and needs during enrollment.

11. A method of finding and enrolling or re-enrolling a person into Medicaid, using telephonic, computer and electronic communications means, comprising the steps of:
communicating with a candidate or person assumed to meet requirements for admission to Medicaid via a telephone conversation, a questionnaire on a mobile device or a questionnaire on a personal computer and if communication is not made, automatically using a search program and computer to find personal contact information of the candidate to then reattempt communication therewith;
storing the personal contact information of the candidate in a secured rewritable and searchable computer database having a calendar;
utilizing the personal contact information to communicate with the candidate;
determining if the candidate is eligible for Medicaid, and if so, renaming the candidate an applicant;
directing the applicant to an appropriate enrollment means for providing information for enrollment in Medicaid;
securing enrollment data, wherein securing enrollment data can be started on any one of a telephone conversation, a questionnaire on a mobile device or a questionnaire on a personal computer and completed, later, on any one or more of a telephone conversation, a questionnaire on a mobile device or a questionnaire on a personal computer, from the applicant, and storing the enrollment data in the secured rewritable and searchable database, and associating the data with the personal contact information for the applicant as applicant data;
automatically, by allowing the computer to use the applicant data, merging the applicant data to a precise computer chosen application form to create an application for enrollment in the specific desirable program and submitting the application form for enrollment; and
updating the applicant data and associating the data with the calendar to automatically contact the applicant, using the updated applicant data, within a specified time period for re-enrollment.

12. The method of claim 11, wherein a computer includes any electronic data entry and manipulation devices including one or more of a personal mobile device, a mobile telephone, an electronic pad, an electronic tablet, a personal computer, a laptop computer or any other data entry device and components thereof, including software and peripherals.

13. The method of claim 11, wherein enrollment data is created by the applicant answering a series of questions, the answers being automatically recorded in the secure database.

14. The method of claim 11, wherein when an attempt to communicate with a candidate is unsuccessful, the process automatically uses means to search public and private databases to find updated contact information and then uses the updated contact information to again attempt to communicate with the candidate; this process continuing until communication is made.
